(19) United States
(12) Patent Application Publication
(10) Pub. No.: US 2023/0220038 A1
(43) Pub. Date: Jul. 13, 2023

(54) BRI2 BRICHOS DOMAIN FOR DELIVERY OF PROTEINS INTO CNS NEURONS

(71) Applicant: ALPHABETA AB, Djursholm (SE)

(72) Inventor: Jan JOHANSSON, Stockholm (SE)

(21) Appl. No.: 18/046,138

(22) Filed: Oct. 12, 2022

Related U.S. Application Data
(63) Continuation-in-part of application No. PCT/EP2021/059434, filed on Apr. 12, 2021.

(51) Int. Cl. C07K 14/705

(52) U.S. Cl. CPC C07K 14/705; A61K 38/00

(57) ABSTRACT

A facilitated method for use in efficient delivery of a pharmaceutical composition over a tissue such as the blood brain barrier and into neurons in the CNS for the treatment of Alzheimer's disease in a mammal, including man is provided. The method is comprising the steps of administrating a therapeutically effective amount of the isolated recombinant Bri2 BRICHOS and lipid microbubbles and enables an increased amount of the isolated recombinant protein to reach the brain to efficiently combat Aβ42 neurotoxicity.

Furthermore, a method for use in enhanced delivery of proteins comprising Bri2 BRICHOS and variants thereof over the blood-brain barrier, facilitating treatment and/or diagnostics of Alzheimer's disease and other neurological diseases is described herein. The method is comprising the steps of administrating a therapeutically effective amount of proteins comprising Bri2 BRICHOS and variants thereof as a first protein moiety coupled to another (non-Bri2) second protein or polypeptide moiety and optionally lipid microbubbles, in the absence of ultrasound treatment.

### CROSS-REFERENCE TO RELATED APPLICATIONS

This application is a continuation-in-part of International Application No. PCT/EP2021/059434, filed on Apr. 12, 2021, which application is incorporated by reference herein.

### INCORPORATION BY REFERENCE OF SEQUENCE LISTING PROVIDED AS AN XML FILE

A Sequence Listing is provided herewith as a Sequence Listing XML, “AWAP-043CIP_SequenceListing” created on Oct. 11, 2022 and having a size of 16 kilobytes. The contents of the Sequence Listing XML are incorporated by reference herein in their entirety.

### FIELD OF THE INVENTION

The present invention pertains to the field of medicine. More specifically, this invention relates to use of substances and agents for improved medical treatment of Alzheimer's disease, in mammals, such as man. The invention furthermore relates to substances and agents for transporting a protein across the blood-brain barrier in a mammal, including man. The invention also relates to delivery of proteins into CNS neurons and treatment of neurological diseases.

### BACKGROUND TO THE INVENTION

An increasing number of neurodegenerative conditions are linked to protein misfolding and aggregation, such as Alzheimer's disease and familial British or Danish dementia. These diseases are characterized by protein deposits in the brain parenchyma and cerebral arteries, and occur in inherited and sporadic forms. Even though these diseases have different clinical symptoms, they share some common pathological features, such as neuronal loss, protein aggregates, and presence of tau tangles. From a biochemical point of view, the proteins involved have a tendency to form β-sheet structures and are prone to aggregate into amyloid fibrils. Alzheimer's disease and familial British or Danish dementia display several similar neuropathological hallmarks. Amyloid plaques, neurofibrillary tangles, Congophilic amyloid angiopathy and neurodegeneration are observed. Alzheimer's disease is one of the most common causes of dementia in man. It is a chronic and fatal disease associated with neural cell degeneration in the brain of the affected individual, characterized by the presence of amyloid plaques consisting of extracellular deposits of amyloid β-peptide (Aβ-peptide). The neural cell atrophy caused by Aβ aggregation results in deficiency of acetylcholine and other signalling substances. It is known that Aβ-peptide, having 40-42 amino acid residues, is produced by processing of the amyloid precursor protein (APP, 695-770 amino acid residues), which is a type I membrane protein normally expressed by the neurons of the central nervous system, but the reasons for this processing are incompletely understood. The released Aβ peptide contains a part of the transmembrane region of APP (Aβ residues 29-40/42) and includes a discordant helix, i.e. a helix composed of amino acids with a high propensity to form β-strands. Aβ is prone to misfold and aggregate when removed from its stabilising membrane environment.

Bri2 (SEQ ID NO: 1, also referred to as integral membrane protein 2B, ITM2B), is a 266-residue type II membrane protein (FIG. 1) with ubiquitous expression, whose function and folded structure are unknown. Bri2 is proteolytically cleaved at three locations; cleavage by furin in the C-terminal region generates a 23-residue peptide (ABri23), processing of the ectodomain by ADAM10 results in release of the BRICHOS domain from the membrane-bound N-terminal part, and intramembrane cleavage by SPPL2a/2b liberates the intracellular domain. Familial British and Danish dementia are caused by mutations in the Bri2 gene that result in a loss of a stop codon, which in turn results in two different 11-residue extensions of the C-terminal part, and, after furin cleavage, generation of 34-residue peptides (ABri and ADan, respectively) instead of the normally released ABri23. The longer peptides are prone to aggregation into amyloid fibrils and deposition in brain tissue or cerebral vessels, with concomitant neuronal loss and dementia.

Recent studies have shown that Bri2 and Aβ co-localize in amyloid plaques in brain parenchyma and vessels, suggesting that the proteins interact at some stage during misfolding and aggregation. Using transfected cell lines, Bri2 has been found to interact with APP, and to modulate APP processing by increasing β-secretase generated fragments. Generation of a fusion protein containing Bri2 and Aβ40 indicates that the Bri protein can affect Aβ aggregation properties, and using a transgenic mouse model, ABri23 has been proposed to interact with Aβ42 and prevent its aggregation (Kim et al. J. Neurosci. 28: 6030-6036 (2008); WO 2009/009396). It has also been suggested that Aβ production can be reduced or prevented by a protein containing the first 102 amino acid residues of Bri2 (WO 2006/138355).

The BRICHOS domain is a naturally occurring chaperone with anti-amyloid properties found in 10 different human proprotein families, one (proSP-C) of which is associated with amyloid lung disease, and one (Bri2/ITM2b) that is as earlier described associated with the amyloid related dementias familial British or Danish dementia. Recombinant human (rh) BRICHOS domains from proSP-C and Bri2 delay Aβ40 and Aβ42 fibril formation and reduce the neurotoxicity associated with Aβ42 fibril formation in vitro and in vivo (WO 2011/62655).

The blood-brain barrier (BBB) functions to maintain a delicate homeostasis required for proper neuronal function. The BBB also functions as a barrier towards substances and agents targeting the brain, larger molecules are unable to spontaneously cross the BBB. The BBB nevertheless presents an efficient pathway for the transportation of compositions such as agents, drugs and biologic drugs, such as proteins, into the central nervous system (CNS). Only small and lipophilic molecules have been shown to be able to pass passively across the BBB. Recent studies have shown that isolated recombinant Bri2 BRICHOS proteins with an attached AU1 tag for immunodetection (Bri2 BRICHOS-AU1) can be detected in the brain parenchyma after peripheral (intravenous) administration to wild type mice, while recombinant proSP-C BRICHOS proteins only pass into the cerebrospinal fluid (CSF). This suggests that an unknown mechanism for transport of Bri2 BRICHOS over the BBB exists (Mikitsh et al. Perspect Medicin Chem. 6:11-24 (2014); Sanchez-Covarrubias et al. Curr Pharm Des. 20: 1422-49 (2014); Tambaro et al. J Biol Chem. 294: 2606-2615 (2019)).

Various methods have been developed for delivery of compositions over the BBB. Focused ultrasound combined with intravenous lipid microbubbles (MBs) results in a localized and reversible opening of the BBB, which allows transport of macromolecules including proteins into the brain parenchyma surrounding the area that is targeted by ultrasound (Mikitsh et al. Perspect Medicin Chem. 6:11-24 (2014); Brasnjevic et al. Prog Neurobiol. 87: 212-51 (2009); Konofagou et al. Theranostics. 2: 1223-37 (2012); Sierra et al. J Cereb Blood Flow Metab. 37:1236-1250 (2017)). These methods require specialized ultrasound equipment. It has also been considered that the ultrasound treatment may under certain conditions give rise to vascular damage.

Current therapeutic approaches for treatment of Alzheimer's disease are mainly directed to treating the symptoms and include cholinergic replacement therapy, e.g. inhibition of acetylcholinesterase, small inhibitors that interact with soluble Aβ oligomers, and so-called β-sheet breakers that prevent elongation of already formed β-sheet structures. Furthermore, it is of interest to be able to lower the doses of the therapeutic drugs in order to reduce side-effects associated with ultrasound treatments in combination with other treatments.

### SUMMARY OF THE INVENTION

It is an object of the invention to provide a new treatment option for the treatment of Alzheimer's disease in a mammal, including man.

It is also an object of the invention to provide a robust and facilitated method and means for efficient delivery of proteins, such as therapeutic agents, over the blood brain barrier and into the CNS. In particular, it is an object of the invention to achieve delivery of proteins, such as therapeutic agents, into CNS neurons.

One object of the invention is to provide a simple and efficient method and means for efficient delivery of Bri2 BRICHOS and variants thereof over the blood-brain barrier in the treatment of Alzheimer's disease.

It is another object of the invention to decrease the tendency of proteins that are prone to fibrillate to aggregate into amyloid fibrils, or even prevent proteins that are prone to fibrillate from aggregating into amyloid fibrils.

It is yet another object to decrease formation of amyloid plaques consisting of extracellular deposits in the brain of a mammal of proteins that are prone to fibrillate.

It is an object of the invention to impinge on the distribution of isolated recombinant Bri2 BRICHOS and variants thereof so that the therapeutically effective amount of isolated recombinant Bri2 BRICHOS reaching the brain is increased to efficiently combat Aβ42 neurotoxicity.

It is another object of the invention to provide a protein and a method for improved treatment, in vivo diagnostics and prognostics, and/or imaging of Alzheimer's disease and other neurological diseases in a mammal, including man.

The present invention is generally based on the insight that the isolated recombinant protein Bri2 BRICHOS and variants thereof can be efficiently delivered over the blood-brain barrier when administered in combination with lipid microbubbles and/or nanodroplets, without any step of ultrasound treatment of any tissue of a mammal. It is surprising that co-administration with lipid microbubbles and/or nanodroplets achieves an improved uptake of Bri2 BRICHOS and variants thereof in absence of ultrasound treatment, compared to both (a) in the absence of lipid microbubbles and/or nanodroplets, and (b) in the presence of ultrasound treatment. The method is not comprising any step of treatment of any tissue of the mammal with optical, audio or ultrasonic waves which make the microbubbles and/or nanodroplets cavitate in the tissue.

Furthermore, the present invention is also based on the insight that microbubbles and/or nanodroplets alone, in the absence of ultrasound treatment, may be used to enhance the delivery of proteins comprising Bri2 BRICHOS and variants thereof as a first protein moiety coupled to another (non-Bri2) second protein or polypeptide moiety over the blood-brain barrier and thereby e.g. facilitate treatment and/or diagnostics of Alzheimer's disease and other neurological diseases involving the second protein or polypeptide moiety.

One aspect of the present invention is based on the insight that proteins comprising the isolated recombinant protein Bri2 BRICHOS and variants thereof, including Bri2 BRICHOS R221E, can be efficiently delivered over the blood-brain barrier into CNS neurons. The method is not requiring any step of administering lipid microbubbles and/or nanodroplets. The method is not requiring any step of treatment of any tissue of the mammal with optical, audio or ultrasonic waves. Bri2 BRICHOS R221E is described in WO 2021/140140 A1, which is incorporated herein in its entirety by reference.

For these and other objects that will be evident from the following description and the appended claims, the present invention provides according to a first aspect an isolated recombinant protein comprising Bri2 BRICHOS and variants thereof for use in a method of treatment of Alzheimer's disease. According to a second aspect, there is provided a method of treating Alzheimer's Disease comprising administrating an isolated recombinant protein comprising Bri2 BRICHOS and variants thereof and lipid microbubbles and/or nanodroplets without any ultrasound treatment.

According to a third aspect, there is provided a protein comprising a first protein moiety which is Bri2 BRICHOS and variants thereof and a second protein or polypeptide moiety, and a combination thereof with lipid microbubbles and/or nanodroplets. According to a fourth aspect, this combination is useful in a method for transporting the protein across the blood-brain barrier in a mammal without any ultrasound treatment.

The third aspect of a protein comprising a first protein moiety which is Bri2 BRICHOS and variants thereof and a second protein or polypeptide moiety is advantageously useful for increasing transport of the second protein or polypeptide moiety over the blood-brain barrier. The first (Bri2 BRICHOS) moiety aids in the transport, and this is achieved without lipid microbubbles and/or nanodroplets. One particularly useful variant of the first protein moiety is then Bri2 BRICHOS R221E since it is demonstrated herein to improve the transport compared to wildtype Bri2 BRICHOS. The transport can be further improved with lipid microbubbles and/or nanodroplets. If lipid microbubbles and/or nanodroplets are included, the method does not require any step of treatment of any tissue of the mammal with optical, audio or ultrasonic waves which make the microbubbles and/or nanodroplets cavitate in the tissue.

### BRIEF DESCRIPTION OF THE DRAWINGS

FIG. 1 shows a schematic outline of Bri2 (SEQ ID NO: 1) and processing sites.

FIG. 2 shows an alignment of some mammalian Bri2 BRICHOS amino acid sequences (SEQ ID NOS: 5-10).

FIG. 3 shows the study design in the Examples.

FIG. 4 shows that Bri2 BRICHOS, but not proSP-C BRICHOS, is detected in non-FUS-targeted brain hemisphere after i.v. injection together with lipid microbubbles and/or nanodroplets.

FIG. 5 shows that Bri2 BRICHOS is detected in both hemispheres after i.v. injection together with lipid microbubbles and/or nanodroplets.

FIG. 6 shows intracellular immunostaining for Bri2 BRICHOS-AU1 in the cortex and hippocampus after i.v. injection together with lipid microbubbles and/or nanodroplets.

FIG. 7 shows western blot detection of Bri2 BRICHOS-AU1 in both brain hemispheres after systemic injection together with lipid microbubbles and/or nanodroplets.

FIG. 8 shows that Rh Bri2 BRICHOS-mCherry is found in the striatum after systemic injection in combination with lipid microbubbles and/or nanodroplets.

FIG. 9 illustrates detection of Bri2 BRICHOS in App^{NL-G-F} and App^{NL-F} mouse brains after repeated injections of Bri2 BRICHOS R221E.

FIG. 10 shows hCMEC/D3 monolayer permeability of different proteins and macromolecules.

FIG. 11 shows staining with anti-NT antibody of brain cortex of mice injected with NT-Bri2 BRICHOS (A) or PBS (B).

FIG. 12 illustrates that Rh Bri2 BRICHOS mediates uptake into mouse primary neurons.

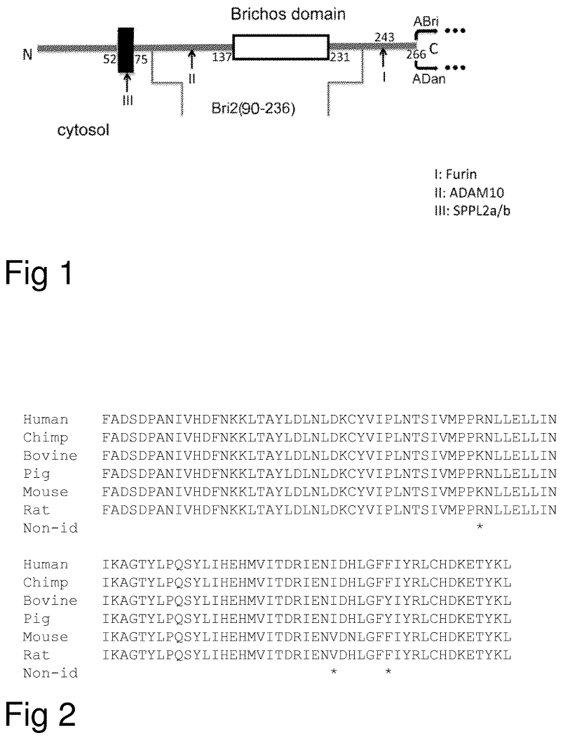

### LIST OF APPENDED SEQUENCES

| | | |
|---|---|---|
| SEQ ID NO: 1 | human Bri2 | MVKVTFNSAL AQKEAKKDEP KSGEEALIIP |
| | | PDAVAVDCKD PDDVVPVGQR RAWCWCMCFG |
| | | LAFMLAGVIL GGAYLYKYFA LQPDDVYYCG |
| | | IKYIKDDVIL NEPSADAPAA LYQTIEENIK |
| | | IFEEEEVEFI SVPVPEFADS DPANIVHDFN |
| | | KKLTAYLDLN LDKCYVIPLN TSIVMPPRNL |
| | | LELLINIKAG TYLPQSYLIH EHMVITDRIE |
| | | NIDHLGFFIY RLCHDKETYK LQRRETIKGI |
| | | QKREASNCFA IRHFENKFAV ETLICS |
| SEQ ID NO: 2 | human Bri2 | QTIEENIKIF EEEEVEFISV PVPEFADSDP |
| | (113-231) | ANIVHDFNKK LTAYLDLNLD KCYVIPLNTS |
| | | IVMPPRNLLE LLINIKAGTY LPQSYLIHEH |
| | | MVITDRIENI DHLGFFIYRL CHDKETYKL |
| SEQ ID NO: 3 | human Bri2(1-89) | MVKVTFNSAL AQKEAKKDEP KSGEEALIIP |
| | | PDAVAVDCKD PDDVVPVGQR RAWCWCMCFG |
| | | LAFMLAGVIL GGAYLYKYFA LQPDDVYYC |
| SEQ ID NO: 4 | human ABri23 | EASNCFAIRH FENKFAVETL ICS |
| | [Bri2(244-266)] | |
| SEQ ID NO: 5 | human Bri2_{BRICHOS} | FADSDPANIV HDFNKKLTAY LDLNLDKCYV |
| | [Bri2(137-231)] | IPLNTSIVMP PRNLLELLIN IKAGTYLPQS |
| | | YLIHEHMVIT DRIENIDHLG FFIYRLCHDK |
| | | ETYKL |
| SEQ ID NO: 6 | chimpanzee | FADSDPANIV HDFNKKLTAY LDLNLDKCYV |
| | Bri2_{BRICHOS} | IPLNTSIVMP PRNLLELLIN IKAGTYLPQS |
| | | YLIHEHMVIT DRIENIDHLG FFIYRLCHDK |
| | | ETYKL |
| SEQ ID NO: 7 | bovine Bri2_{BRICHOS} | FADSDPANIV HDFNKKLTAY LDLNLDKCYV |
| | | IPLNTSIVMP PKNLLELLIN IKAGTYLPQS |
| | | YLIHEHMVIT DRIENIDHLG FYIYRLCHDK |
| | | ETYKL |
| SEQ ID NO: 8 | pig Bri2_{BRICHOS} | FADSDPANIV HDFNKKLTAY LDLNLDKCYV |
| | | IPLNTSIVMP PRNLLELLIN IKAGTYLPQS |
| | | YLIHEHMVIT DRIENIDHLG FYIYRLCHDK |
| | | ETYKL |
| SEQ ID NO: 9 | mouse Bri2_{BRICHOS} | FADSDPANIV HDFNKKLTAY LDLNLDKCYV |
| | | IPLNTSIVMP PKNLLELLIN IKAGTYLPQS |
| | | YLIHEHMVIT DRIENVDNLG FFIYRLCHDK |
| | | ETYKL |
| SEQ ID NO: 10 | rat Bri2_{BRICHOS} | FADSDPANIV HDFNKKLTAY LDLNLDKCYV |
| | | IPLNTSIVMP PRNLLELLIN IKAGTYLPQS |
| | | YLIHEHMVIT DRIENVDHLG FFIYRLCHDK |
| | | ETYKL |
| SEQ ID NO: 11 | rh Bri2 BRICHOS-AU1 | GSQTIEENIK IFEEEEVEFI SVPVPEFADS |
| | | DPANIVHDFN KKLTAYLDLN LDKCYVIPLN |
| | | TSIVMPPRNL LELLINIKAG TYLPQSYLIH |
| | | EHMVITDRIE NIDHLGFFIY RLCHDKETYK |
| | | LDTYRYI |
| SEQ ID NO: 12 | rh Bri2 BRICHOS-mCherry | GSQTIEENIK IFEEEEVEFI SVPVPEFADS |
| | | DPANIVHDFN KKLTAYLDLN LDKCYVIPLN |
| | | TSIVMPPRNL LELLINIKAG TYLPQSYLIH |
| | | EHMVITDRIE NIDHLGFFIY RLCHDKETYK |
| | | LGGGGGGSVS KGEEDNMAII KEFMRFKVHM |
| | | EGSVNGHEFE IEGEGEGRPY EGTQTAKLKV |
| | | TKGGPLPFAW DILSPQFMYG SKAYVKHPAD |
| | | IPDYLKLSFP EGFKWERVMN FEDGGVVTVT |
| | | QDSSLQDGEF IYKVKLRGTN FPSDGPVMQK |
| | | KTMGWEASSE RMYPEDGALK GEIKQRLKLK |
| | | DGGHYDAEVK TTYKAKKPVQ LPGAYNVNIK |
| | | LDITSHNEDY TIVEQYERAE GRHSTGGMDE |
| | | LYK |
| SEQ ID NO: 13 | rh Bri2 BRICHOS | QTIEENIKIF EEEEVEFISV PVPEFADSDP |
| | R221E | ANIVHDFNKK LTAYLDLNLD KCYVIPLNTS |
| | | IVMPPRNLLE LLINIKAGTY LPQSYLIHEH |
| | | MVITDRIENI DHLGFFIYEL CHDKETYKL |
| SEQ ID NO: 14 | human Bri2_{BRICHOS} | FADSDPANIV HDFNKKLTAY LDLNLDKCYV |
| | [Bri2(137-231)] | IPLNTSIVMP PRNLLELLIN IKAGTYLPQS |
| | R221E | YLIHEHMVIT DRIENIDHLG FFIYELCHDK |
| | | ETYKL |

### DETAILED DESCRIPTION OF THE INVENTION

Bri2 (SEQ ID NO: 1), also referred to as integral membrane protein 2B (ITM2B), contains an evolutionary conserved BRICHOS domain spanning residues 137-231 (SEQ ID NO: 5). BRICHOS domains are found in more than 10 different protein families that are functionally unrelated and expressed in different tissues. The name BRICHOS refers to identification of the domain in Bri, chondromodulin-1 related to chondrosarcoma and in lung surfactant protein C precursor (proSP-C) involved in respiratory disease.

Proteins comprising the BRICHOS domain of a mammalian Bri2 (ITM2B) and structurally similar proteins have the capacity to decrease amyloid fibril formation and aggregation of Aβ-peptide and ABri/ADan peptides.

The blood-brain barrier (BBB) functions to maintain a delicate homeostasis required for proper neuronal function, the BBB also functions as a barrier towards substances and agents targeting the brain, larger molecules are unable to spontaneously cross the BBB. The BBB nevertheless presents an efficient pathway for the transportation of pharmaceutical compositions as proteins into the central nervous system (CNS). Only small and lipophilic molecules have been shown to be able to pass passively across the BBB.

The present invention is generally based on the insight that an isolated Bri2 BRICHOS protein as such, or coupled to a second protein or polypeptide moiety, can be efficiently delivered into the brain in combination with lipid microbubbles and/or nanodroplets and would thus, be useful for treating Alzheimer's disease.

Furthermore, an isolated Bri2 BRICHOS protein coupled to a second protein or polypeptide moiety, can be efficiently delivered into the brain, even without lipid microbubbles and nanodroplets.

The lipid microbubbles and/or nanodroplets and the isolated protein may be administered in combination. The term “combination” and/or “combination”, as used herein refers to that the isolated proteins and the lipid microbubbles and/or nanodroplets may be administered individually in any order independently of each other. The term may also refer to that the recombinant proteins and the lipid microbubbles and/or nanodroplets may be administered individually and at the same time. Furthermore, the term may refer to that the isolated proteins and lipid microbubbles and/or nanodroplets may be administered in the same pharmaceutical composition.

The isolated protein according to the invention can be incorporated into pharmaceutical compositions. Such compositions typically include the isolated protein according to the invention and a suitable pharmaceutically acceptable carrier. As used herein, a “suitable pharmaceutical carrier” includes solvents, dispersion media, coatings, isotonic and absorption delaying agents, and the like, that are compatible with pharmaceutical administration. Supplementary active compounds can also be incorporated into the compositions.

In prior art methods, ultrasound is an important local stimulus for triggering drug release at the target tissue and may consist of pressure waves at frequencies of 20 kHz or greater. Like optical and audio waves, ultrasonic waves are focused, reflected, and refracted through a medium.

Microbubbles and/or nanodroplets in combination with ultrasound treatment are typically used in medical diagnostics and non-invasive delivering of pharmaceutical compositions and genes to different tissues. Focused ultrasound combined with intravenously administered microbubbles and/or nanodroplets is a technology that has been shown in multiple in vivo models to efficiently deliver small- and large-molecules over the BBB and to a specifically targeted brain region in a minimally invasive way. This technique involves administration of lipid-based microbubbles and/or nanodroplets together with the pharmaceutical composition to be delivered. The ultrasonic waves make the microbubbles and/or nanodroplets cavitate within the capillaries in the brain. At these low pressures, microbubbles and/or nanodroplets exhibit stable cavitation which induces an increase in the opening of the BBB. This makes the tight junctions between the endothelial cells loosen transiently, which results in transient and local opening of the BBB. As a result, macromolecules present in the circulation are delivered into the brain parenchyma (Galan-Acosta et al. Mol Cell Neurosci, 103498 (2020)).

Here, we design a facilitated method for efficient delivery of a composition over a tissue such as the blood brain barrier and into neurons in the CNS. This method combines the administration of an isolated protein with the administration of lipid microbubbles and/or nanodroplets. Importantly, the present method does not involve any step of ultrasound treatment. This is advantageous as it does not require any ultrasound equipment. Furthermore, side effects associated with ultrasound treatment, e.g. vascular damage, can be avoided. It is surprising that co-administration with lipid microbubbles and/or nanodroplets achieves an improved uptake of Bri2 BRICHOS and variants thereof in absence of ultrasound treatment, compared to both (a) in the absence of lipid microbubbles and/or nanodroplets, and (b) in the presence of ultrasound treatment.

The method is not comprising any step of treatment of any tissue of the mammal with optical, audio or ultrasonic waves which make the microbubbles and/or nanodroplets cavitate in the tissue.

For these and other objects that will be evident from the following description, the present invention provides according to a first aspect an isolated recombinant protein selected from the group of proteins consisting of an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); with the provisos that said protein is not comprising an amino acid sequence having at least 70% identity to residues 1-89 of Bri2 from human (SEQ ID NO: 3); and said protein is not comprising an amino acid sequence having at least 70% identity to human ABri23 (SEQ ID NO: 4); for use in a method of treatment of Alzheimer's disease in a mammal, including man, in need thereof comprising the steps of;
administrating to said mammal a plurality of lipid microbubbles and/or nanodroplets
administrating to said mammal said isolated recombinant protein; wherein said isolated recombinant protein is not comprised within said microbubbles and/or nanodroplets; and
   wherein the method is not comprising any step of ultrasound treatment of any tissue of the mammal.

It is experimentally shown herein that the lipid microbubbles and/or nanodroplets alone, i.e. in the absence of ultrasound treatment, are able to mediate uniform transfer of Bri2 BRICHOS over the BBB, and that Bri2 BRICHOS is delivered into the brain parenchyma and is efficiently taken up by neurons in the cortex as well as the hippocampus when the administration of Bri2 BRICHOS and lipid microbubbles and/or nanodroplets is combined.

It has surprisingly been found that Bri2 BRICHOS proteins are delivered to wildtype mice brain when administered in combination with microbubbles and/or nanodroplets. This is particularly surprising in view of the various methods that have previously been developed, wherein focused ultrasound combined with intravenous lipid microbubbles and/or nanodroplets are used to deliver drugs and other compositions over the BBB by allowing transport of macromolecules including proteins into the brain parenchyma surrounding the area that is targeted with ultrasound.

The present invention is based on the herein disclosed, surprising insights that the microbubbles and/or nanodroplets on their own, without being combined with any step of ultrasound treatment of any tissue of the mammal have the capacity to mediate increased transfer of the isolated recombinant protein over the BBB. Microbubbles and/or nanodroplets increase the passage of Bri2 BRICHOS over the BBB and uptake in neurons.

This aspect of the invention is advantageous in that transient and local opening of the BBB can be obtained without the use of any additional treatment steps such as ultrasound treatments and additional equipment associated with such additional treatments. Thus, a facilitated method for efficient delivery of a composition over a tissue such as the blood brain barrier and into neurons in the CNS is provided. Another advantage is, as shown herein, that the uptake of the isolated recombinant proteins into the brain parenchyma is highly increased when administered in combination with lipid microbubbles and/or nanodroplets in comparison with administrating the isolated recombinant proteins alone as previously described in WO 2011/162655.

The term “% similarity”, as used throughout the specification and the appended claims, is calculated as described for “% identity”, with the exception that the hydrophobic residues Ala, Val, Phe, Pro, Leu, Ile, Trp, Met and Cys are similar; the basic residues Lys, Arg and His are similar; the acidic residues Glu and Asp are similar; and the hydrophilic, uncharged residues Gln, Asn, Ser, Thr and Tyr are similar. The remaining natural amino acid Gly is not similar to any other amino acid in this context.

Throughout this description, alternative embodiments fulfil, instead of the specified percentage of identity, the corresponding percentage of similarity. Other alternative embodiments fulfil the specified percentage of identity as well as another, higher percentage of similarity, selected from the group of preferred percentages of identity for each sequence. For example, the isolated recombinant protein sequence may be 70% similar to another protein sequence; or it may be 70% identical to another sequence; or it may be 70% identical and furthermore 90% similar to another sequence.

For avoidance of doubt, the amino acid sequence having at least the given identity to residues 113-231 of Bri2 from human or any one of the BRICHOS domains of Bri2 consists of more than or equal to 70, such as more than or equal to 80, such as more than or equal to 90 amino acid residues. A preferable size range is 70-100 amino acid residues, such as 80-100 amino acid residues, e.g. 90-100 amino acid residues.

It is noted that the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10) is highly conserved, see alignment in FIG. 2. Without desiring to be bound to any specific theory, it is contemplated that the BRICHOS domain harbours the desired activity with respect to the Aβ and ABri/ADan peptides. It is preferred that the isolated recombinant protein according to the invention is selected from the group consisting of proteins comprising an amino acid sequence having at least 80%, preferably at least 90%, such as at least 95%, identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10). In a preferred embodiment, the isolated recombinant protein according to the invention contains all amino acid residues that are conserved in the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10), i.e. all amino acid residues of SEQ ID NO: 5 except for residues 42, 76 and 82 (corresponding to residues 178, 212 and 218 in full length Bri2, SEQ ID NO: 1). In specific embodiments, the isolated recombinant protein according to the invention is selected from the group consisting of proteins comprising any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10), i.e. it contains one of these BRICHOS domains, preferably the human BRICHOS domain (SEQ ID NO: 5).

In a preferred embodiment, the amino acid residue corresponding to position 221 in SEQ ID NO: 1 is selected from the group consisting of Glu and Asp. In a specific embodiment, the amino acid residue corresponding to position 221 in SEQ ID NO: 1 is Glu. We mutated rh Bri2 BRICHOS so that the monomer is stabilised relative to larger oligomers and rh Bri2 BRICHOS R221E (SEQ ID NO: 13) selectively reduces Aβ42 oligomer generation and alleviates Aβ42-induced neurotoxicity in hippocampal slice preparations. Rh Bri2 BRICHOS R221E passes the BBB in mice and shows a trend towards higher passage than the wildtype protein. This result is in line with the previous observation that monomeric wildtype rh Bri2 BRICHOS passes the BBB more efficiently than larger oligomers.

In contrast to previous teachings, the isolated recombinant protein according to the invention is not comprising an amino acid sequence having at least 70% identity to residues 1-89 of Bri2 from human (SEQ ID NO: 3). In certain embodiments, the isolated recombinant protein according to the invention is not comprising an amino acid sequence having at least 50% identity to residues 1-89 of Bri2 from human (SEQ ID NO: 3). This implies that the isolated recombinant protein according to the invention contains a core amino acid sequence which displays a high similarity or identity to residues 113-231 of Bri2 from human (SEQ ID NO: 2) and/or a mammalian BRICHOS domain of Bri2 from (SEQ ID NOS: 5-10) and optionally one or more other amino acid sequences, which other amino acid sequences may not display a high similarity or identity to residues 1-89 of Bri2 from human (SEQ ID NO: 3).

For avoidance of doubt, amino acid sequences that are shorter than 10 amino acid residues are not considered relevant in the context of being excluded from the isolated recombinant protein according to the invention. Thus, the isolated recombinant protein according to the invention is not comprising an amino acid sequence that consists of more than or equal to 10 amino acid residues having at least the given identity to residues 1-89 of Bri2 from human (SEQ ID NO: 3).

Furthermore, the isolated recombinant protein according to the invention is not comprising an amino acid sequence having at least 70% identity to residues 244-266 of Bri2 from human, i.e. human ABri23 (SEQ ID NO: 4). In certain embodiments, the isolated recombinant protein according to the invention is not comprising an amino acid sequence having at least 50% identity to residues human ABri23 (SEQ ID NO: 4). As set out above, this implies that the isolated recombinant protein according to the invention contains a core amino acid sequence which displays a high similarity or identity to residues 113-231 of Bri2 from human (SEQ ID NO: 2) and/or a mammalian BRICHOS domain of Bri2 from (SEQ ID NOS: 5-10) and optionally one or more other amino acid sequences, which other amino acid sequences may not display a high similarity or identity to human ABri23 (SEQ ID NO: 4).

For avoidance of doubt, amino acid sequences that are shorter than 10 amino acid residues are not considered relevant in the context of being excluded from the isolated recombinant protein according to the invention. Thus, the isolated recombinant protein according to the invention is not comprising an amino acid sequence that consists of more than or equal to 10 amino acid residues having at least the given identity to human ABri23 (SEQ ID NO: 4).

In preferred embodiments, the isolated recombinant protein for use according to the invention is selected from the group consisting of proteins comprising an amino acid sequence having at least 70% identity to residues 113-231 of Bri2 from human (SEQ ID NO: 2); and proteins comprising an amino acid sequence having at least 70% identity to the BRICHOS domain of Bri2 from human (SEQ ID NO: 5).

In a preferred embodiment an isolated recombinant protein selected from the group of proteins consisting of an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); with the provisos that said protein is not comprising an amino acid sequence having at least 70% identity to residues 1-89 of Bri2 from human (SEQ ID NO: 3); and said protein is not comprising an amino acid sequence having at least 70% identity to human ABri23 (SEQ ID NO: 4); for use in a method of treatment of Alzheimer's disease in a mammal, including man, in need thereof consisting of the steps of;
administrating to said mammal a plurality of lipid microbubbles and/or nanodroplets
administrating to said mammal said isolated recombinant protein;
   wherein said isolated recombinant protein is not comprised within said microbubbles and/or nanodroplets.

In specific embodiments a combination of an isolated recombinant protein and lipid microbubbles and/or nanodroplets for use in a method of treatment of Alzheimer's disease in a mammal, including man, in need thereof is comprising the steps of;

administrating to said mammal a plurality of lipid microbubbles and/or nanodroplets

administrating to said mammal said isolated recombinant protein; wherein said isolated recombinant protein is not comprised within said microbubbles and/or nanodroplets; and

wherein the method is not comprising any step of ultrasound treatment of any tissue of the mammal.

In a specific embodiment a combination of an isolated recombinant protein and lipid microbubbles and/or nanodroplets for use in a method of treatment of Alzheimer's disease in a mammal, including man, in need thereof is consisting of the steps of;
administrating to said mammal a plurality of lipid microbubbles and/or nanodroplets

administrating to said mammal said isolated recombinant protein; wherein said isolated recombinant protein is not comprised within said microbubbles and/or nanodroplets.

The pharmaceutical composition comprising the isolated recombinant protein may be useful as a medicament, specifically in treatment of conditions such as Alzheimer's disease in a mammal, including man.

A pharmaceutical composition is formulated to be compatible with its intended route of administration. Examples of routes of administration include parenteral (e.g. intravenous, intraarterial), intraperitoneal, intramuscular, intradermal and intranasal.

Administration of the microbubbles and/or nanodroplets and the isolated recombinant protein may include injections.

Sterile injectable solutions can be prepared by incorporating the isolated recombinant protein according to the invention in the required amount in an appropriate solvent with one or a combination of ingredients enumerated above, as required, followed by filtered sterilization. Generally, dispersions may be prepared by incorporating the isolated recombinant protein according the invention into a sterile vehicle which contains a dispersion medium and other ingredients required. In the case of sterile powders for the preparation of sterile injectable solutions, the preferred methods of preparation are vacuum drying and freeze-drying, which yield a powder of the isolated recombinant protein according the invention plus any additional desired ingredient from a previously sterile-filtered solution thereof.

Generally, sterile injectable solutions of lipid microbubbles and/or nanodroplets may be prepared, as set out e.g. in Feshitan, J. A. et al., J. Colloid Interface Sci. 329 (2), 316-324 (2009). Briefly, the gas perfluorobutane (PFB) may be used to form the microbubbles and/or nanodroplets. PFB may act as a gas core, that can be introduced in order to activate the lipid microbubbles and/or nanodroplets, and which are later isolated. The microbubbles and/or nanodroplets may be coated with 1,2-Distearoyl-sn-glycero-3-phosphocholine (DSPC) and Polyoxyethylene-40 stearate (PEG40S) DSPC and PEG40S. The obtained dried lipid film may be hydrated with filtered PBS and mixed to a final lipid suspension.

The lipid mixture may be sonicated in order to disperse the lipid aggregates into small, unilamellar liposomes. PFB gas may be introduced by flowing it over the surface of the lipid suspension. Subsequently, higher power sonication may be applied to the suspension at the gas-liquid interface to generate microbubbles and/or nanodroplets. Following isolation, the microbubbles and/or nanodroplets according to the invention may be incorporated into a sterile vehicle e.g. the microbubble and/or nanodroplet suspension may be collected into 30-mL syringes, washing and size fractionating may be achieved by centrifugation in order to collect all microbubbles and/or nanodroplets from the suspension into a cake resting against the syringe plunger. The remaining suspension (infranatant), which may contain residual lipids and vesicles that did not form part of the microbubble shells, may be recycled to produce the next batch of microbubbles and/or nanodroplets. All resulting cakes may be combined and re-suspended in PBS to improve total yield.

In certain embodiments said isolated recombinant protein and said lipid microbubbles and/or nanodroplets are administered intravenously.

When an isolated protein according to the invention is to be administered to an animal (e.g. a human) to treat Alzheimer's disease, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific isolated recombinant protein employed, the age, body weight, general health, gender, and diet of the subject, the time of administration, the route of administration, the rate of excretion, any drug combination, and the degree of expression or activity to be modulated.

Data obtained from cell culture assays and animal studies may be used in formulating a range of dosage for use in humans.

The dosage may vary within this range depending upon the dosage form employed and the route of administration utilized. For any compound used in the method of the invention, the therapeutically effective dose can be estimated initially from cell culture assays in which, e.g. the rate of fibril formation or the rate of cell death is observed. A dose may be formulated in animal models to achieve a circulating plasma concentration range that includes the IC₅₀ (i. e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of an isolated recombinant protein according to the invention (i. e., an effective dosage) ranges from about 1 to 50 mg/kg body weight. In one embodiment, a therapeutically effective amount of at least 1 mg/kg, such as at least 5 mg/kg, more preferably such as at least 10 mg/kg of said isolated recombinant protein is administered.

In another embodiment, a therapeutically effective amount of less than 50 mg/kg, such as less than 30 mg/kg, more preferably less than 20 mg/kg of the isolated recombinant protein is administered.

The isolated recombinant protein can be administered over an extended period of time to the subject, e.g., over the subject's lifetime. A dosage of 1 mg/kg to 50 mg/kg body weight is usually appropriate. By administrating the isolated recombinant protein in combination with the lipid microbubbles and/or nanodroplets according to the invention administration of significantly lower doses of the isolated recombinant protein than previously described in WO 2011/162655 is enabled. The administration of lipid microbubbles and/or nanodroplets results in the localized and reversible opening of the BBB, which allows for efficient transport and increased uptake of the isolated recombinant protein into the brain parenchyma. This aspect of the invention is also advantageous since the risk of side effects associated with administration of a therapeutic composition for both prophylactic and therapeutic methods of treating a subject who has or is at risk of (or susceptible to) Alzheimer's disease is minimized.

In some cases, the lipid microbubbles and/or nanodroplets and the isolated recombinant protein can be administered once per week for between about 1 to 10 weeks, preferably between 2 to 8 weeks, more preferably between about 3 to 7 weeks, and even more preferably for about 4, 5, or 6 weeks. The compound can also be administered chronically. The skilled person will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount lipid microbubbles and/or nanodroplets and the isolated recombinant protein can include a single treatment or, preferably, can include a series of treatments.

In further embodiments, the isolated protein is selected from the group consisting of proteins comprising an amino acid sequence having at least 70%, preferably at least 75%, 80%, 85%, 90%, 95%, or 99% identity to residues 113-231 of Bri2 from human (SEQ ID NO: 2); and proteins comprising an amino acid sequence having at least 70%, preferably at least 75%, 80%, 85%, 90%, 95%, or 99% identity to the BRICHOS domain of Bri2 from human (SEQ ID NO: 5).

In preferred embodiments, the isolated protein is selected from the group consisting of residues 113-231 of Bri2 from human (SEQ ID NO: 2); and the BRICHOS domain of Bri2 from human (SEQ ID NO: 5).

According to a related aspect, the present invention provides an isolated selected from the group of proteins comprising an amino acid sequence having at least 70%, preferably at least 75%, 80%, 85%, 90%, 95%, or 99% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).

In another embodiment the isolated protein is selected from the group consisting of proteins comprising any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).

In specific embodiments, the isolated protein is selected from the group consisting of any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).

In certain embodiments, the isolated protein consists of less than or equal to 200 amino acid residues, such as less than or equal to 150 amino or even 100 amino acid residues. In certain embodiments, the isolated protein consists of more than or equal to 90 amino acid residues, such as more than or equal to 100 amino acid residues.

A preferable size range of the isolated protein is 80-200 amino acid residues, such as 90-150 amino acid residues, e.g. 90-100 amino acid residues.

Microbubbles and/or nanodroplets are small gas-filled microspheres. They may consist of gas surrounded by a by a lipid, lipopolymer, or polymer shell. They may also be similar in size to red blood cells and may range from 0.5-10 μm.

The gas-filled microbubbles and/or nanodroplets, oscillate and vibrate when a sonic energy field is applied. Microbubbles and/or nanodroplets having a hydrophilic outer layer to interact with the bloodstream and a hydrophobic inner layer to house the gas molecules are the most thermodynamically stable. Air, sulfur hexafluoride, and perfluorocarbon gases may serve as the composition of the microbubble interior. For increased stability and persistence in the bloodstream, gases with high molecular weight as well as low solubility in the blood are attractive candidates for microbubble gas cores. Microbubbles and/or nanodroplets may be used for drug delivery, and they may not only serve as drug vehicles but also as a means to permeate otherwise impenetrable barriers, specifically the blood brain barrier.

In further embodiments of the invention the microbubbles and/or said nanodroplets are lipid coated.

As disclosed herein, the microbubbles and/or nanodroplets may comprise 1,2-distearyol-sn-glycero-3-phosphocoline (DSPC), 1,2-distearyol-sn-glycero-3-phosphoethanolamine-N-(metoxy(polyethyleneglycol)2000) and a gas core of perfluorobutane. The microbubbles and/or nanodroplets may e.g. comprise sulphur hexafluoride, polyethylene glycol (PEG, Macrogol), distearylphosphatidylcholine (DSPC), sodium 1,2-dipalmitoyl-sn-glycero-3-phosphatidylglycerol and palmitic acid.

In a preferred embodiment of the invention the individual microbubbles and/or nanodroplets according to the invention have a diameter in the range of 1-8 μm, such as 2-6 μm, such as 4-5 μm.

The skilled person will appreciate that the embodiments discussed above in relation to the first aspect of the present disclosure, are equally relevant and applicable to the second, third and further aspects disclosed herein. This particularly applies to embodiments relating to the isolated recombinant protein and the lipid microbubbles and/or nanodroplets, the inventive combination underlying the efficient and uniform transfer of the recombinant isolated protein over the BBB and into the brain parenchyma, as well as the efficient uptake by the neurons in the cortex and the hippocampus, and to embodiments relating to the mode and route of administration. For the sake of brevity these will not be repeated here or will only be briefly mentioned.

In a second aspect of the invention, there is provided a method of treating Alzheimer's disease in a mammal, including man, in need thereof comprising the steps of;
administrating to said mammal a plurality of lipid microbubbles and/or nanodroplets
administrating to said mammal an isolated recombinant protein selected from the group of proteins consisting of an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); with the provisos that said protein is not comprising an amino acid sequence having at least 70% identity to residues 1-89 of Bri2 from human (SEQ ID NO: 3); and said protein is not comprising an amino acid sequence having at least 70% identity to human ABri23 (SEQ ID NO: 4);
   wherein said isolated recombinant protein is not comprised within said microbubbles and/or nanodroplets; and wherein the method is not comprising any step of ultrasound treatment of any tissue of the mammal.

In embodiments of the second aspect, a method of treating Alzheimer's disease in a mammal, including man, in need thereof is provided, consisting of the steps of;
administrating to said mammal a plurality of lipid microbubbles and/or nanodroplets
administrating to said mammal an isolated recombinant protein selected from the group of proteins consisting of an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); with the provisos that said protein is not comprising an amino acid sequence having at least 70% identity to residues 1-89 of Bri2 from human (SEQ ID NO: 3); and said protein is not comprising an amino acid sequence having at least 70% identity to human ABri23 (SEQ ID NO: 4);
   wherein said isolated recombinant protein is not comprised within said microbubbles and/or nanodroplets.

As a further aspect of the invention, there is provided an isolated recombinant protein consisting of residues 113-231 of Bri2 from human (SEQ ID NO: 2.

The present invention provides for both prophylactic and therapeutic methods of treating a subject at risk of (or susceptible to) Alzheimer's disease. As used herein, the term “treatment” is defined as the application or administration of an isolated recombinant protein and lipid microbubbles and/or nanodroplets according to the invention to a patient, or application or administration of an isolated recombinant protein and lipid microbubbles and/or nanodroplets according to the invention to an isolated tissue or cell line from a patient, who has Alzheimer's disease, a symptom of disease or a predisposition toward a disease, with the purpose to cure, heal, alleviate, relieve, alter, remedy, ameliorate, improve or affect the disease, the symptoms of disease or the predisposition toward disease. In specific embodiments, the treatment is selected from the group consisting of preventive, palliative and curative treatment.

In one aspect, the invention provides a method for preventing a disease or condition (i. e., decreasing the risk of contracting, or decreasing the rate at which symptoms appear that are associated with a disease or condition) associated with fibril formation caused by Aβ peptide and/or ABri/ADan peptide by administering to the subject an isolated recombinant protein and lipid microbubbles and/or nanodroplets according to the invention that reduces aggregation of the polypeptide. Subjects at risk for Alzheimer's disease can be identified by, for example, any or a combination of appropriate diagnostic or prognostic assays known in the art. Administration of a prophylactic agent can occur prior to the manifestation of symptoms characteristic of the disease, such that the disease is prevented or, alternatively, delayed in its progression.

The isolated recombinant protein and the lipid microbubbles and/or nanodroplets according to the invention can be administered to a patient at therapeutically effective doses to prevent, treat or ameliorate disorders involving fibril formation associated with Alzheimer's disease. A therapeutically effective dose refers to that amount of the compound sufficient to result in amelioration of symptoms of the disorders. Toxicity and therapeutic efficacy of such compounds can be determined by standard pharmaceutical procedures as described above.

According to a different aspect of the invention, the Bri2 BRICHOS protein and variants thereof are useful in the delivery of protein or polypeptides such as therapeutic agents, antibodies and protein tags by providing distinct advantage of improving for example therapeutic potential of drugs and drug targeting, in vivo diagnostics and prognostics, and in vivo imaging. A protein may thus comprise at least one further protein or polypeptide moiety, wherein the protein moiety may be a biological polymer, an oligomer and an oligopeptide such as peptides and polypeptides.

As a third aspect of the invention, there is provided an isolated protein comprising

(i) a first protein moiety selected from the group of proteins comprising an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); and

(ii) a second protein or polypeptide moiety, preferably containing at least 50 amino acid residues;

wherein said isolated protein is not comprising an amino acid sequence having at least 70% identity to residues 1-89 of Bri2 from human (SEQ ID NO: 3); and
wherein said isolated protein is not comprising an amino acid sequence having at least 70% identity to human ABri23 (SEQ ID NO: 4).

Proteins comprising the first protein moieties are unique in their in vivo therapeutic application by providing tissue-specific targeting and/or release of drugs for use in treatment and therapies, in vivo diagnostics and prognostics, and in vivo imaging when used in combination with microbubbles and/or nanodroplets.

The Bri2 BRICHOS domain provides the capacity to transport the second protein or polypeptide moiety across the blood-brain barrier in a mammal without any ultrasound treatment. An isolated protein comprising a Bri2 BRICHOS domain and second protein or polypeptide moiety such as protein drugs, polypeptide drugs, protein tags, fluorescent proteins, antibodies, enzymes and/or neurotrophic factors is advantageous since it facilitates and enhances the treatment of Alzheimer's Disease and other neurological diseases.

In an embodiment, the first protein moiety of the isolated protein is selected from the group of proteins comprising an amino acid sequence having at least 70%, preferably at least 80%, 85%, 90%, 95% or 99%, identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).

In another embodiment, the first protein moiety is selected from the group of proteins comprising any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).

In one embodiment of the invention the first protein moiety of the isolated protein is selected from the group of proteins comprising an amino acid sequence having at least 70%, preferably at least 80%, 85%, 90%, 95% or 99%, identity to any one of residues 113-231 of Bri2 from human (SEQ ID NO: 2) and the BRICHOS domain of Bri2 from human (SEQ ID NO: 5).

In a further embodiment the first protein moiety is selected from the group consisting of residues 113-231 of Bri2 from human (SEQ ID NO: 2); and the BRICHOS domain of Bri2 from human (SEQ ID NO: 5).

In one embodiment, the amino acid residue in the first protein moiety corresponding to position 221 in SEQ ID NO: 1 is selected from the group consisting of Glu and Asp, preferably Glu. We mutated rh Bri2 BRICHOS so that the monomer is stabilised relative to larger oligomers and rh Bri2 BRICHOS R221E (SEQ ID NO: 13-14) selectively reduces Aβ42 oligomer generation and alleviates Aβ42-induced neurotoxicity in hippocampal slice preparations. Rh Bri2 BRICHOS R221E passes the BBB in mice and shows a trend towards higher passage than the wildtype protein. This result is in line with the previous observation that monomeric wildtype rh Bri2 BRICHOS passes the BBB more efficiently than larger oligomers. The improved propensity of the R221D/E variant for passage is particularly advantageous when efficiently delivery into the brain is desired, without co-administration of lipid microbubbles and nanodroplets.

In one embodiment, the first protein moiety is consisting of less than or equal to 200 amino acid residues, such as less than or equal to 150 amino acid residues. In one embodiment, the first protein moiety is consisting of more than or equal to 90 amino acid residues.

Generally, delivery of large therapeutics molecules into the brain to treat central nervous system (CNS) diseases is a major drug development challenge. The BBB serves to restrict movement of substances from the circulating blood to the CNS. Thus, only approximately 0.1% of circulating antibodies cross the intact BBB, severely limiting the therapeutic utility of antibody therapeutics for CNS disorders. Furthermore, the BBB excludes from the brain 100% of large-molecule neurotherapeutics and more than 98% of all small-molecule drugs.

In one embodiment, the isolated protein does not contain a cleavage site between the first protein moiety and the Bri2-BRICHOS sequence and the second protein or polypeptide moiety. In another embodiment, the isolated protein contains a cleavage site between the first protein moiety and the Bri2-BRICHOS sequence and the second protein or polypeptide moiety, e.g. the cleavage site in Bri2 which in the native protein is naturally cleaved by proprotein convertases to release Abri peptide.

An object of the invention is to provide an isolated protein wherein the second protein or polypeptide moiety of the isolated protein contains from 50 to 2000 amino acid residues, such as from 50 to 1000 amino acid residues, such as from 50 to 500 amino acid residues, such as from 50 to 100 amino acid residues. In another object of the invention the size of the second protein or polypeptide moiety of the isolated protein is 5-200 kDa, such as 5-100 kDa, such as 5-50 kDa, such as 5-10 kDa.

The inventors have surprisingly found that an isolated protein comprising Bri2 BRICHOS and a second protein or polypeptide moiety can cross the BBB despite its large size.

In some embodiments of the invention, the first protein moiety of the isolated protein is linked directly or indirectly to the amino-terminal or the carboxy-terminal end of the second protein or polypeptide moiety.

In another embodiment of the invention the second protein or polypeptide moiety of the isolated protein constitutes the amino-terminal and/or the carboxy-terminal end of the isolated protein.

As previously mentioned, the BBB often hinders the brain delivery of large therapeutic molecules such as protein drugs, polypeptide drugs, protein tags, fluorescent proteins, antibodies, enzymes and/or neurotrophic factors.

Protein and polypeptide drugs can be used to replace a protein that is abnormal or deficient in a particular disease. They can also augment the body's supply of a beneficial protein to help reduce the impact of diseases treatments. Examples of common and widely used protein drugs are insulin, Interferon alpha and Interleukin-2.

Protein tags are peptide sequences grafted onto a protein, e.g. genetically grafted onto a recombinant protein. Examples of protein tags include solubilization tags, epitope tags and fluorescence tags.

Solubilization tags are used, especially for recombinant proteins expressed in chaperone-deficient species such as E. coli, to assist in the proper folding in proteins and keep them from precipitating.

Epitope tags are short peptide sequences which are chosen because high-affinity antibodies can be reliably produced in many different species.

Fluorescence tags and proteins are used to give visual readout on a protein. They may be used to tag components in a cell, a tissue or an organ so they can be studied using fluorescence spectroscopy, fluorescence microscopy and other imaging techniques. GFP and its variants are the most commonly used fluorescence tags. mCherry on the other hand is a member of the mFruits family of monomeric red fluorescent proteins (mRFPs) and belongs to the group of fluorescent protein chromophores used as instruments to visualize genes and analyze their functions in experiments.

The second protein moiety can also increase the stability of the isolated protein and the first protein moiety which itself may be useful as a therapeutical agent, e.g. increase half-life in the body. Thereby, the frequency of treatments may be decreased. One possibility is that the second protein moiety is an Fc moiety, i.e. the fragment crystallizable region (Fc region) from the tail region of an antibody that interacts with cell surface receptors called Fc receptors and some proteins of the complement system.

In another embodiment, the second protein or polypeptide moiety of the isolated protein is selected from the group consisting of protein drugs, polypeptide drugs, protein tags, fluorescent proteins, antibodies, enzymes and/or neurotrophic factors.

Antibodies, also known as an immunoglobulin (Ig) are large, Y-shaped proteins used by the immune system to identify and neutralize foreign objects such as pathogenic bacteria and viruses. Using their binding mechanism antibodies are widely used in therapies, wherein they are employed to treat diseases such as rheumatoid arthritis, multiple sclerosis, psoriasis, and many forms of cancers. Monoclonal antibodies that have been studied and/or are used for treatment of Alzheimer's disease are aducanumab, gantenerumab, 3D6 (bapineuzumab) and m266 (solanezumab).

In a preferred embodiment, the second protein or polypeptide moiety of the isolated protein is an antibody.

Neurotrophic factors (NTFs) are a family of biomolecules, nearly all of which are peptides or small proteins that support the growth, survival, and differentiation of both developing and mature neurons. Neurotrophic factors also promote the initial growth and development of neurons in the central nervous system and peripheral nervous system, and they are capable of regrowing damaged neurons in test tubes and animal models. Some neurotrophic factors are also released by the target tissue in order to guide the growth of developing axons. In studies, neurotrophic factors are normally used in conjunction with other techniques, neurotrophic factors may be immobilized to a scaffold structure. In neural drug delivery systems, they are loosely immobilized such that they can be selectively released at specified times and in specified amounts.

In an embodiment, the second protein or polypeptide moiety of the isolated protein is a neurotrophin selected from the group consisting of brain-derived neurotrophic factor (BDNF), nerve growth factor (NGF), neurotrophin-3, neurotrophin-4, ciliary neurotrophic factor (CNTF), glial cell line-derived neurotrophic factor (GDNF), ephrins, epidermal growth factor (EGF), transforming growth factor (TGF), insulin-like growth factor (IGF), vascular endothelial growth factor (VEGF), fibroblast growth factor (FGF), platelet-derived growth factor (PDGF), and/or interleukins.

Enzymes are proteins that act as biological catalysts and accelerate chemical reactions. Enzymes are required for many chemical interconversions that support life and speed up all the biochemical processes in the body. These characteristics distinguish them from other types of drugs. Due to these characteristics, enzymes are widely used medically either alone or adjunctly with other therapies, with the purpose of safe treatment of various diseases. Examples of therapeutic enzymes used for treatment and different therapies of various disorders are α-L-iduronidase, Iduronate sulfatase, N-acetylgalactosamine 6-sulfatase, N-acetylgalactosamine 4-sulfatase, α-galactosidase, α-glucosidase, β-glucocerebrosidase and/or Lysosomal acid lipase. Further examples of therapeutic enzymes which can constitute the second protein include enzymes known to be genetically mutated, and therefore functionally deficient in lysosomal storage diseases (LSDs).

In another embodiment, the second protein or polypeptide moiety of the isolated protein is selected from the group consisting of α-L-iduronidase, Iduronate sulfatase, N-acetylgalactosamine 6-sulfatase, N-acetylgalactosamine 4-sulfatase, α-galactosidase, α-glucosidase, β-glucocerebrosidase and/or Lysosomal acid lipase.

Fusion proteins are proteins created through the joining of two or more genes that originally coded for separate proteins. Translation of these fusion genes results in a single protein with functional properties derived from each of the original proteins. Recombinant fusion proteins are created artificially by recombinant DNA technology for use in biological research or therapeutics.

In some embodiments of the invention the isolated protein is a recombinant fusion protein.

Chemical linking is the process of chemically joining two or more protein molecules such as chemically linking a first protein moiety to a second protein or polypeptide moiety. Chemical linking enables attachment of a moiety such as a protein, polypeptide, protein drug, tag and fluorescent molecules to another protein molecule in order to transport to and target cells and tissues, providing target specific therapy and treatments, and/or improved in vivo diagnostics and prognostics, imaging and aid in detection of a molecule(s).

In another embodiment of the invention the first protein moiety of the isolated protein is chemically linked to said second protein or polypeptide moiety.

As a further aspect of the invention, there is provided a combination of an isolated protein according as disclosed above and a plurality of lipid microbubbles and/or nanodroplets, wherein the isolated protein is not comprised within said microbubbles and/or said nanodroplets.

In one embodiment, a kit comprising an isolated protein as disclosed above and a plurality of lipid microbubbles and/or nanodroplets is provided, wherein the isolated protein is not comprised within said microbubbles and/or said nanodroplets.

Brain-penetrating molecules, recombinant proteins and/or biologics are generally not successful unless the pharmaceutical crosses the BBB. Thus, the BBB drug delivery is the limiting factor in the future development of new therapeutics for the brain.

The inventors have designed a facilitated method for efficient delivery of a composition over a tissue such as the BBB and into neurons in the CNS.

For these and other objects, the present invention provides according to a further aspect of the invention a method for transporting an isolated protein as disclosed herein across the blood-brain barrier in a mammal, including man, in need thereof consisting of the steps of;
administrating to said mammal a plurality of lipid microbubbles and/or nanodroplets; and
administrating to said mammal said isolated protein.

In a specific embodiment the method does not comprise any step of ultrasound treatment of any tissue of the mammal. The present invention provides according to a yet further aspect an isolated protein as disclosed herein for use in a method of treatment involving transporting said isolated protein across the blood-brain barrier in a mammal, including man, in need thereof comprising the steps of:

administrating to said mammal a plurality of lipid microbubbles and/or nanodroplets;
administrating to said mammal said isolated protein;
wherein said isolated protein is not comprised within said microbubbles and/or said nanodroplets.

In a certain embodiment, the method is not comprising any step of ultrasound treatment of any tissue of the mammal.

The method is not comprising any step of treatment of any tissue of the mammal with optical, audio or ultrasonic waves which make the microbubbles and/or nanodroplets cavitate in the tissue.

According to another aspect, a method for transporting an isolated protein as disclosed herein across the blood-brain barrier in a mammal, including man, in need thereof is comprising, or consisting of, the step of;
administrating to said mammal said isolated protein.

In a specific embodiment, there is provided an isolated protein as disclosed herein for use in a method of treatment involving transporting said protein across the blood-brain barrier in a mammal, including man, in need thereof consisting of the steps of:
administrating to said mammal a plurality of lipid microbubbles and/or nanodroplets; and
administrating to said mammal said isolated protein;
   wherein said isolated protein is not comprised within said microbubbles and/or said nanodroplets.

It has surprisingly been found that both rh Bri2 BRICHOS fused to a short peptide tag or to a globular protein were detected in the brain parenchyma 2 hours after administration together with lipid microbubbles and/or nanodroplets. Analyses of the brain parenchyma by immunohistochemistry and western blot clearly show similar distribution of delivered protein in both hemispheres, with strong staining in the cortex, hippocampus and the choroid plexus after administration of rh Bri2 BRICHOS-AU1 with microbubbles and/or nanodroplets. The internalization by cells of rh Bri2 BRICHOS-AU1 delivered together with microbubbles and/or nanodroplets was seen, which is consistent with results obtained from FUS-mediated delivery of rh Bri2 BRICHOS-AU1 (Galan-Acosta et al., Mol Cell Neurosci. 2020:103498). Bri2 is expressed not only in the CNS but also in peripheral tissues, and the existence of a transport system that allows the crosstalk between both sites could be envisioned.

The transport of drugs into cerebrospinal fluid (CSF) cannot be extrapolated to BBB passage, and in line with that notion it can be noted that another BRICHOS protein, rh proSP-C BRICHOS, was detected in CSF after intravenous injection in mice, but was not found in brain homogenates. This indicates that passage of the blood CSF barrier does not ensure transport over the BBB or into the brain parenchyma (Tambaro et al. J Biol Chem. 294: 2606-2615 (2019)). The permeability of the BBB differs between different brain regions, for example in areas close to the choroid plexus and in the circumventricular areas the permeability is higher than in the rest of the brain.

Moreover, quantitative analysis of total rh Bri2 BRICHOS domain that reached the brain parenchyma by sandwich ELISA shows considerable amounts of rh Bri2 BRICHOS-AU1 in both hemispheres. According to this analysis, 1% of the totally injected rh Bri2 BRICHOS domain administered with lipid microbubbles and/or nanodroplets is detected in the brain 2 hours after injection, which is between 2-10 fold more than the passage observed when rh Bri2 BRICHOS-AU1 was administered without microbubbles and/or nanodroplets (Tambaro et al., J Biol Chem. 2019; 294(8):2606-15) or with microbubbles and/or nanodroplets plus FUS (Galan-Acosta et al., Mol Cell Neurosci. 2020:103498). In the case of the rh Bri2 BRICHOS-mCherry fusion protein, the magnitude of the increased BBB passage mediated by microbubbles and/or nanodroplets was not quantified, but the results indicate an effect as the fusion protein could only be detected in the brain when it was administered together with microbubbles and/or nanodroplets.

Taken together, the current data demonstrate that microbubbles and/or nanodroplets significantly increase the passage of rh Bri2 BRICHOS over the BBB, also when it is fused to a large, 30 kDa globular protein. Incorporation or association of drugs with lipid nanoparticles can increase bioavailability, but in the present invention the rh Bri2 BRICHOS proteins and microbubbles and/or nanodroplets are administered independently, making it unlikely that rh Bri2 BRICHOS passes the BBB together with microbubbles and/or nanodroplets. Without desiring to be bound by any particular theory, the inventors suspect that increased passage of rh Bri2 BRICHOS, or rh Bri2 BRICHOS-mCherry, over the BBB in the presence of microbubbles and/or nanodroplets is mediated by a prolonged half-life of rh Bri2 BRICHOS in the circulation. Other means to increase the half-life of rh Bri2 BRICHOS in the circulation will most likely increase its passage over the BBB and rh Bri2 BRICHOS fused to other proteins, including for example antibodies, enzymes or neurothrophic factors may increase their passage into the brain parenchyma, as functional mCherry now was shown to be able to be transported into the brain of wildtype mice.

The findings of the present invention show that the extent of rh Bri2 BRICHOS passage through the BBB into the brain parenchyma in the presence of microbubbles and/or nanodroplets is higher (1% of injected dose) than what have been observed for peripherally administered antibodies (about 0.1% of injected dose) (Bard et al. Nat Med. 2000; 6(8):916-919 and Zuchero et al. 2016; 89(1):70-82). Rh Bri2 BRICHOS BBB passage is likewise apparently more efficient than affibodies designed to improve CNS uptake, which were only detected in low amounts in the cerebrospinal fluid after injection (Meister et al. Int J Mol Sci. 2020; 21(8)).

As set out above, proteins comprising the isolated recombinant protein Bri2 BRICHOS and variants thereof, including Bri2 BRICHOS R221E, can be efficiently delivered over the blood-brain barrier into CNS neurons. The method is not requiring any step of administering lipid microbubbles and/or nanodroplets, The method is not requiring any step of treatment of any tissue of the mammal with optical, audio or ultrasonic waves. Thus, the isolated Bri2 BRICHOS protein coupled to a second protein or polypeptide moiety, can be efficiently delivered into the brain, even without lipid microbubbles and nanodroplets.

As demonstrated herein, an isolated protein comprising a first protein moiety which is a Bri2 BRICHOS protein or a variant thereof and at least one further protein or polypeptide moiety is useful in a method for transporting the isolated protein across the blood-brain barrier in a mammal, including man, in need thereof comprising, or consisting of, the step of:
administrating to said mammal said isolated protein.

Furthermore, the isolated protein comprising a first protein moiety which is a Bri2 BRICHOS protein or a variant thereof and at least one further protein or polypeptide moiety is useful in a method of treating a medical condition in a mammal, including man, in need thereof comprising, or consisting of, the step of:
administrating to said mammal said isolated protein.

Accordingly, the isolated protein is useful as a medicament. The isolated protein is useful in a method of treating a medical condition in a mammal, including man, in need thereof comprising, or consisting of, the step of:
administrating to said mammal said isolated protein.

In these methods, the isolated protein comprising a first protein moiety which is a Bri2 BRICHOS protein or a variant thereof and at least one further protein or polypeptide moiety can be efficiently delivered into the brain, even without lipid microbubbles and nanodroplets.

It is also contemplated that the isolated proteins according to the invention can be administrated by gene therapy, such as by using expression vectors, plasmids or viruses to transfect cells in the neural system, preferably brain, such that the isolated protein is expressed by these cells in the central neural system. This is useful for the treatment of Alzheimer's disease. This may also be useful for treatment of other neurological diseases.

The present invention will now be further illustrated by the following non-limiting examples.

### EXAMPLES

The Examples demonstrate that microbubbles can be used to enhance the delivery of Bri2 BRICHOS and fusion proteins thereof over the BBB and facilitate treatment of AD and other neurological diseases.

### Expression and Purification of Recombinant Proteins

Rh Bri2 BRICHOS domain, corresponding to residues 113-231 of the full length Bri2 protein, was linked to an AU1 tag or the mCherry protein at the C-terminal end. The BRICHOS containing proteins Bri2 BRICHOS-AU1 (SEQ ID NO: 11) and Bri2 BRICHOS-mCherry (SEQ ID NO: 12) were expressed in E. coli and purified. Before injection into mice, rh Bri2 BRICHOS-AU1 was dialyzed (6-8 kDa membrane, Spectrum lab) against filtered and autoclaved phosphate-buffer saline (PBS), pH 7.4. Endotoxins were eliminated by passing the proteins over a Pierce High-Capacity endotoxin removal column (Thermo scientific). The final protein preparations were filtered through a 0.22 μm Millex-GV filter (Millipore Ltd.) and they were stored at −20° C. and thawed a few minutes before injections.

As a control, a proSP-C BRICHOS domain (proSP-C residues 59-197) was produced as set out in Galan-Acosta et al., Mol Cell Neurosci. 2020:103498.

### Animals

Female and male C57BL/6J, age 4-6 months old, weight 24-30 g, were used. All animals were kept on 12-hour light-dark cycles and grouped in cages of 5 mice with food and water available ad libitum. All the experiments were approved and conducted in accordance with the ethical committee of Södra Stockholms Djurförsöksetiska Nämnd (dnr 03049), Linköpings Etiska Nämnd (ID855) or under the guidelines of Columbia University Institutional Animal Care and Use Committee.

### Experimental Design

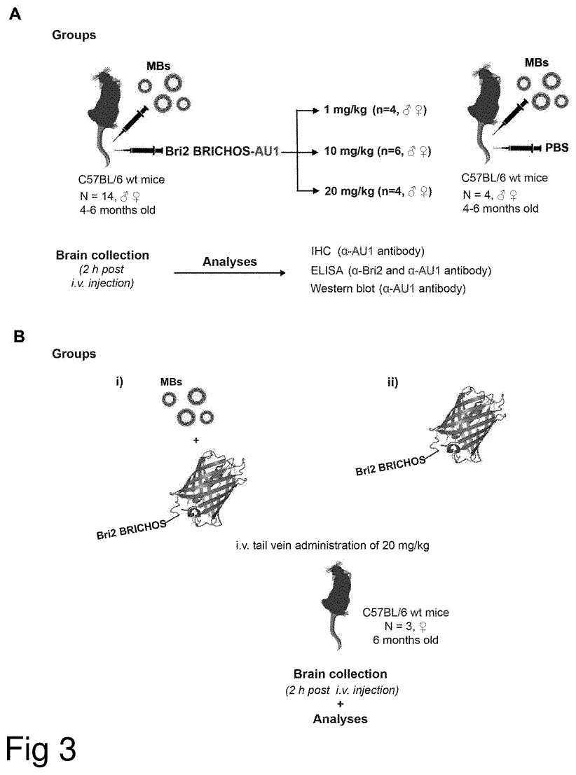

FIG. 3 shows the study design in the Examples. (A) Mice received microbubbles and 1, 10, or 20 mg/kg rh Bri2 BRICHOS-AU1 (SEQ ID NO: 11), or PBS for controls, double i.v. injections in the tail vein. (B) Mice received 20 mg/kg of (i) rh Bri2 BRICHOS-mCherry (SEQ ID NO: 12) and microbubbles, or (ii) rh Bri2 BRICHOS-mCherry only.

Two different types of commercially available microbubbles, either Definity® (Lantheus Medical Imaging, MA, USA) or SonoVue® (Bracco, Milan, Italy) were administered at a dose of 5 μl/g body weight. For one part of the study (FIG. 3A), a total of 14 mice were divided into four groups that received, respectively, i.v. injections of microbubbles and PBS (n=4), microbubbles and 1 (n=4), 10 (n=6) or 20 (n=4) mg/kg body weight of rh Bri2 BRICHOS-AU1 (SEQ ID NO: 11). In the second part of the study (FIG. 3B), a total of 3 mice were administered 20 mg/kg rh Bri2 BRICHOS-mCherry (SEQ ID NO: 12) (n=1) or microbubbles+20 mg/kg rh Bri2 BRICHOS-mCherry (n=2) intravenously. Two mice that received 10 mg/kg of rh Bri2 BRICHOS-AU1 were injected with Definity® microbubbles, the rest of the mice were administered with SonoVue® microbubbles.

All animals received two i.v. injections into the lateral tail vein by using a 29-gauge needle. Prior administration, microbubbles vials were activated with mechanical agitation for 20 seconds and the microbubbles solution was injected in the tail vein over 60 seconds, immediately followed by slow injections of rh Bri2 BRICHOS proteins or PBS controls. Before injections, mice were anesthetized using 2-4% isoflurane (carried with 2% oxygen) and put under a heat lamp in order to dilate the tail veins. Two hours after the injections, the mice were anesthetized and transcardially perfused with 120 ml PBS and one mouse, that was administered Definity® and 10 mg/kg of rh Bri2 BRICHOS-AU1, was perfused with PBS for 5 minutes followed by perfusion with 4% paraformaldehyde (PFA) for 7 minutes. The brains were extracted from skull and either snap-frozen in dry iced and stored at −80° C. or post-fixed in 4% PFA for 48 hours before being embedded in paraffin.

### Antibodies

For immunohistochemistry (IHC), polyclonal rabbit anti-AU1 (Abcam Cat #ab3401) primary antibodies were used at 1:200 dilution, and anti-rabbit secondary antibody conjugated with horseradish peroxidase (HRP) (GE Healthcare Cat #NA934) were diluted to 1:2000. For western blot, primary antibody was polyclonal rabbit anti-AU1 (Abcam Cat #ab3401) at 1:600 dilution and fluorescently labelled secondary anti-rabbit (Li-Cor, Cat #926-32213) antibody was used at 1:10,000 dilution. For sandwich ELISA, goat anti-Bri2 BRICHOS antibody was used for capture at 1:250 dilution, and polyclonal rabbit anti-AU1 (Abcam Cat #ab3401) antibodies were used for detection at 1:2000 dilution.

A rabbit anti-proSP-C antibody was used as previously reported in Galan-Acosta et al., Mol Cell Neurosci. 2020:103498.

### Immunohistochemical Analysis (IHC)

Coronal sections, 5 μm thickness, of paraffin embedded tissue were placed onto Superfrost Plus microscope glass slides (Thermo Scientific) and were let dry at room temperature (RT) overnight to remove residual water. Sections were de-paraffinized by washing in xylene and re-hydrated in decreasing concentrations of ethanol (from 99% to 70%). Sections were pressure boiled in a Decloaking Chamber (Biocare Medical) immersed in DIVA decloaker 1× solution (Biocare Medical, Concord, USA) at 110° C. for 30 min, or incubated in a water bath heated at 95° C. for 30 min. Slides were let cool down at RT for 20 min, then washed with PBS buffer containing 0.1% tween 20 (PBST) and incubated with peroxidase blocking solution (Dako) for 5 min. The sections were washed in Tris-buffered saline (TBS) and additional blocking was performed with Background punisher (Biocare) for 10 min. Primary antibodies diluted in DAKO (Agilent) antibody diluent were incubated for 45 min at RT. Slides were then washed in TBS and incubated with Mach 2 Double stain 2 containing alkaline phospatase (AP) conjugated secondary anti-rabbit antibody for 30 min at RT. AP staining was detected with permanent red (Biosite). Sections were counterstained with hematoxylin (Mayer), de-hydratated through ethanol (from 70% to 99%), cleared in xylene and mounted with DEPEX mounting media (Merck).

### Microscopy

Stained sections were visualized with a Nikon Eclipse E800M optical microscope with Plan-Apochromate objective of 10× and 20× magnifications. A Nikon fluorescence microscope was used to detect rh Bri2 BRICHOS-mCherry proteins in brain samples and recorded with a 20× objective.

### Western Blotting

Brain tissue from one mouse treated with rh Bri2 BRICHOS-AU1+microbubbles and one negative control were homogenized in 50 mM Tris-HCl (pH 7.4), 150 mM NaCl, 1% (v/v) Triton X-100, 0.1% (w/v) SDS and 10 mM EDTA supplemented with protease inhibitor cocktail (Roche, Indianapolis, Ind.). Homogenates were centrifuged at 14,000 rpm (20,800×g) for 30 min at 4° C., and the supernatant was collected and stored at −20° C. Protein concentration was measured by BCA method. The homogenates were diluted in homogenization buffer and 1×SDS reducing buffer (containing 2-mercaptoethanol) so that 100 μg total protein were loaded per sample and well. The samples were heated at 97° C. for 10 min and separated on 4-20% precast polyacrylamide gels (Bio-Rad) and blotted on a nitrocellulose (GE Healthcare) membrane. After blotting, the membranes were blocked using 5% skim milk prepared in 0.1% Tween/TBS for 1 h at RT. Thereafter they were rinsed with 0.1% Tween/TBS and primary antibody diluted in 0.1% Tween/TBS was added over night at 4° C. The membranes were washed three times with 0.1% Tween/TBS and then incubated with secondary antibody prepared in 0.1% Tween/TBS for 1 hat RT. After washing away unbound secondary antibody with 0.1% Tween/TBS, images were acquired using a fluorescence imaging system (Li-Cor, Odyssey CLx).

### Sandwich ELISA

96-well plates (Nunc MicroWell™) were coated with anti Bri2 BRICHOS capture antibody diluted in coating buffer (50 mM carbonate pH 9.6) and incubated overnight at 4° C. After washing three times in 0.05% Tween/PBS, 1% BSA/PBS was used for blocking for 1 hour. Following the washing and blocking steps, the brain samples (250 μg/ml) and standards diluted in 0.05% Tween/PBS were incubated for 2 hours at RT. The plates were washed and rabbit anti-AU1 primary antibody diluted in 0.05% Tween/PBS was added over night at 4° C. The plates were washed three times and secondary anti-rabbit antibody diluted in 0.05% Tween/PBS was added for 2 hours at RT. After washing, tetramethylbenzidine (TMB) (Thermo Fisher) solution was added and incubated for 30 min in darkness. The reaction was stopped by adding stop solution for TMB substrates (Thermo Fisher) and absorbance was measured at 450 nm, using the values for brain homogenates from the non-treated mouse as blank. The standard curve was obtained from rh Bri2 BRICHOS-AU1 protein ranging from 0.1 to 64 ng.

Calculation of concentrations and total amounts of rh Bri2 BRICHOS-AU1 in each brain hemisphere was assessed by assuming a brain density of 1.04 mg/ml, an average brain hemisphere weight of 225 mg and average of total protein in brain hemisphere homogenates of 25.6 mg.

### Results

Passage of Rh Bri2 BRICHOS, but not proSP-C BRICHOS Over the BBB in the Non-Sonicated Hemisphere in the Presence of Lipid Microbubbles

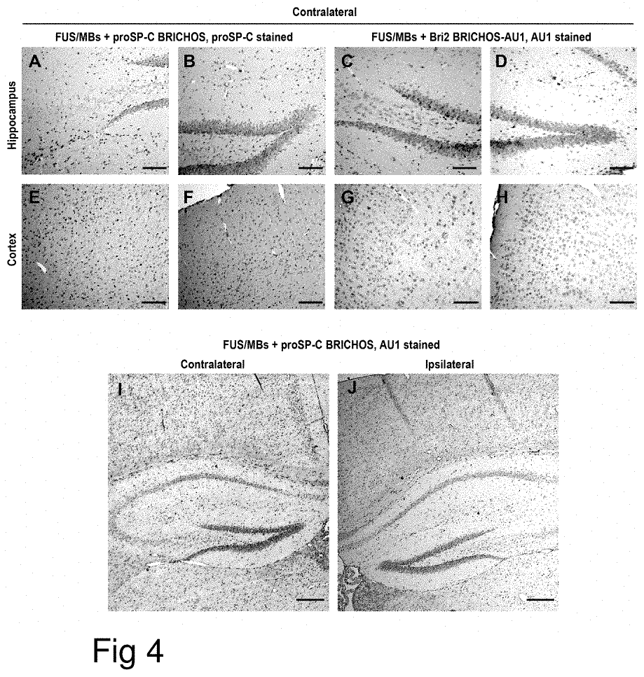

The presence of rh Bri2 BRICHOS-AU1 (SEQ ID NO: 11) in the brain parenchyma was studied. FIG. 4 demonstrates that rh Bri2 BRICHOS, but not rh proSP-C BRICHOS, is detected in non-FUS-targeted brain hemisphere after i.v. injection together with lipid microbubbles. Immunohistochemistry of cortical and hippocampal sections from mice treated with rh proSP-C BRICHOS+microbubbles (A-B, E-F, I-J) or rh Bri2 BRICHOS-AU1+microbubbles (C-D, G-H), from contralateral, non FUS-targeted hemisphere (A-H). (I-J) controls showing no staining for rh Bri2 BRICHOS-AU1 in contralateral or ipsilateral sides of mice treated with intravenous proSP-C BRICHOS+microbubbles and FUS. Tissues were stained using a rabbit anti-proSP-C antibody (A-B, E-F) or a rabbit anti-AU1 antibody (C-D, G-H) followed by AP conjugated secondary antibody and developed with permanent red AP solution. All samples were counterstained with hematoxylin. Scale bars are 100 μm in A-H, and 200 μm in I-J.

Rh Bri2 BRICHOS-AU1 was thus found in cortex and hippocampus of the non FUS-targeted, contralateral hemisphere 2 hours after i.v. administration of 10 mg/kg of rh Bri2 BRICHOS-AU1 in the presence of microbubbles to a wild type mouse (FIG. 4C-D, G-H). Rh proSP-C BRICHOS could not be observed in the non-sonicated hemisphere 2 hours after i.v. injection of 10 mg/kg rh proSP-C BRICHOS and microbubbles in a FUS treated wild type mouse (FIG. 4A-B, E-F). Immunohistochemical staining was negative for rh Bri2 BRICHOS-AU1 in brain sections of a mouse treated with FUS+microbubbles and administered with 10 mg/kg of rh proSP-C BRICHOS, 2 hours after injections, showing that immunoreactivity with anti-AU1 antibody is not an artefact caused by injection of any recombinant protein (FIG. 4I-J).

### Microbubbles Increase BBB Permeability of Rh Bri2 BRICHOS in Wild Type Mice

The effects of microbubbles alone, i.e. without any application of FUS, on rh Bri2 BRICHOS permeability over the BBB were further studied by administering rh Bri2 BRICHOS domain with either an AU1 tag (rh Bri2 BRICHOS-AU1; SEQ ID NO: 11) or mCherry protein (rh Bri2 BRICHOS-mCherry; SEQ ID NO: 12) genetically linked to the C-terminus. SonoVue® microbubbles (dose given 5 μl/g body weight) and different doses of rh Bri2 BRICHOS-AU1 were individually injected into the lateral tail vein of adult wild type mice, and after 2 hours brains were perfused and collected for analyses. Control mice were administered with SonoVue® microbubbles and PBS. The presence of rh Bri2 BRICHOS-AU1 in the brain parenchyma was studied by immunohistochemistry and western blot, while the amounts of rh Bri2 BRICHOS-AU1 were assessed by sandwich ELISA (FIG. 3A).

In order to study the effects of microbubbles on BBB permeability of rh Bri2 BRICHOS fused to a folded globular protein, rh Bri2 BRICHOS-mCherry (44 kDa total molecular weight) was chosen as it can be detected by the fluorescence properties of folded mCherry, which remain unchanged also in fusion with rh Bri2 BRICHOS. In this case, i.v. injections of SonoVue® and 20 mg/kg of rh Bri2 BRICHOS-mCherry (SEQ ID NO: 12) was applied to two wild type mice, while control mice received rh Bri2 BRICHOS-mCherry only (FIG. 3B). Brains were collected 2 hours after injections and analysed macroscopically and under a fluorescence microscope.

The inventors have surprisingly found that microbubbles alone increase passage of rh Bri2 BRICHOS-AU1 over the BBB.

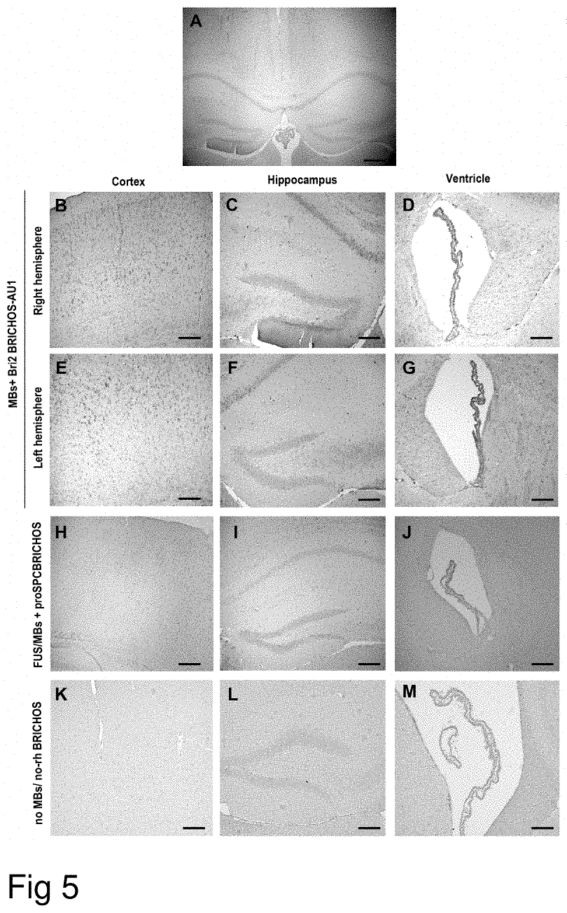

FIG. 5 shows that Bri2 BRICHOS is detected in both hemispheres after i.v. injection together with lipid microbubbles. (A-G) Images from cortex, hippocampus and lateral ventricles from a mouse treated with microbubbles+10 mg/kg rh Bri2 BRICHOS-AU1. (H-J) Images from a mouse injected with 10 mg/kg rh proSP-C BRICHOS and microbubbles. (K-M) Images from a control mouse that received no injections. All sections were incubated with an anti-AU1 antibody followed by an anti-rabbit antibody conjugated with AP and developed with permanent red AP solution, and counterstained with hematoxylin. Sizes of scale bars are 500 μm in panel A and 200 μm in panels B-M

Rh Bri2 BRICHOS-AU1 was detected in the cortex, hippocampus and in the choroid plexus in the lateral ventricles by immunohistochemistry using an antibody against the AU1 tag, after i.v. injection of microbubbles and rh Bri2 BRICHOS-AU1 at the dose of 10 mg/kg (FIG. 5A-G). No differences in staining intensity were seen between the two hemispheres (FIG. 5A-G).

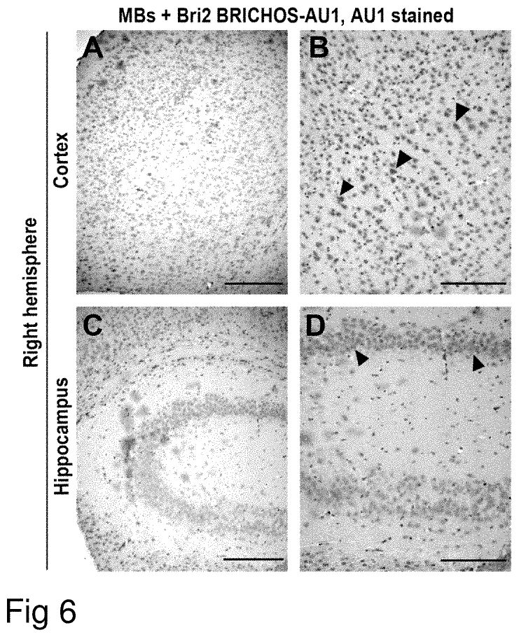

FIG. 6 demonstrates that intracellular immunostaining for rh Bri2 BRICHOS-AU1 is observed in the cortex and hippocampus after i.v. injection together with lipid microbubbles. Images show cortex (A,B) and hippocampus (C,D) of the right hemisphere from a mouse treated with microbubbles+10 mg/kg rh Bri2 BRICHOS-AU1 (SEQ ID NO: 11). Sections were stained for rh Bri2 BRICHOS-AU1 using anti-AU1 antibody and counterstained with hematoxylin. The arrowheads in (B) and (D) panels indicate intracellular staining for rh Bri2 BRICHOS-AU1. Sizes of scale bars are 100 μm for panels A,C; and 50 μm for panels B,D.

Intracellular staining was thus observed in cells present in the cortex and in the hippocampus (FIG. 6A-D). Control brains from mice either injected with rh proSP-C BRICHOS or with PBS were stained in the same way with the anti-AU1 antibody but showed no immunoreactivity (FIG. 5H-M).

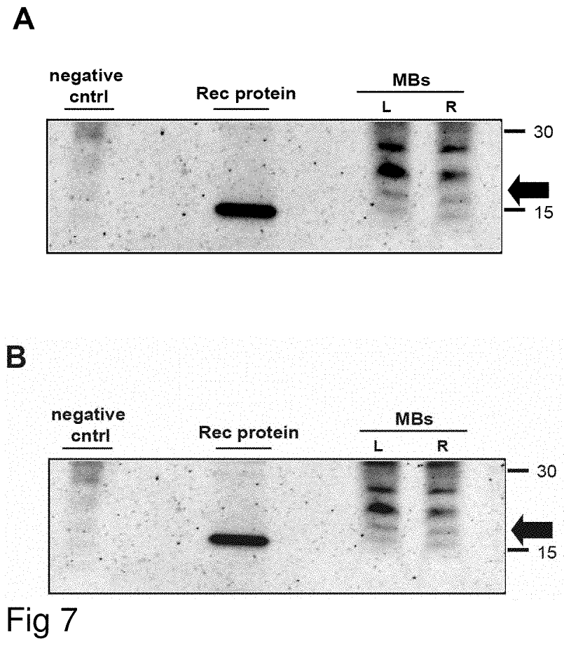

Furthermore, homogenates of both hemispheres from the mouse administered with microbubbles plus 10 mg/kg rh Bri2 BRICHOS-AU1 were analysed by western blot using both anti-AU1 and anti-Bri2 antibodies, which revealed bands migrating as rh Bri2 BRICHOS-AU1 and somewhat slower, while the corresponding bands were absent in a control sample. FIG. 7 shows western blot detection of rh Bri2 BRICHOS-AU1 (SEQ ID NO: 11) in both brain hemispheres after systemic injection together with lipid microbubbles. Western blot analysis of left (L) and right (R) hemispheres from one mouse collected two hours after i.v. injection of microbubbles+rh Bri2 BRICHOS-AU1, and a brain homogenate from a non-treated control mouse (negative control). Rh Bri2 BRICHOS-AU1 is detected using anti-AU1 antibody (A) and an anti-Bri2 antibody (B), as indicated by arrows on the right side of the gel. Lanes marked Rec protein contain 5 ng of rh Bri2 BRICHOS-AU1.

In agreement with immunohistochemistry and western blot results, analyses of homogenates of both hemispheres of the microbubbles plus 10 mg/kg rh Bri2 BRICHOS-AU1 treated mouse 2 hours after injection by sandwich ELISA showed that the rh Bri2 BRICHOS-AU1 concentration in the left hemisphere was 390 nM while in the right hemisphere it was 250 nM, which together correspond to 1% of the total amount administered. Interestingly, higher amounts of isolated recombinant Bri2 BRICHOS-AU1 had reached the brain parenchyma after administration with lipid microbubbles, without ultrasound treatment, compared to without administration of microbubbles. About 1% of the total isolated recombinant Bri2 BRICHOS-AU1, administered at a dose of 10 mg/kg together with lipid microbubbles was detected in the brain 2 hours after intravenous injection, while between 0.1 and 1% (mean 0.5%) was detected after administration of isolated recombinant Bri2 BRICHOS-AU1 at a dose of 20 mg/kg without microbubbles. This is surprising because microbubbles alone (without ultrasound treatment) have not yet been described to increase passage of a protein over the BBB or affect uptake into neurons.

The inventors have furthermore found that rh Bri2 BRICHOS-mCherry passes into the brain parenchyma of wild type mice after injection with, but not without, lipid microbubbles. The BBB permeability of rh Bri2 BRICHOS-mCherry in wild type mice injected with rh Bri2 BRICHOS-mCherry with or without microbubbles was evaluated by observing brains macroscopically and brain sections under a fluorescence microscope.

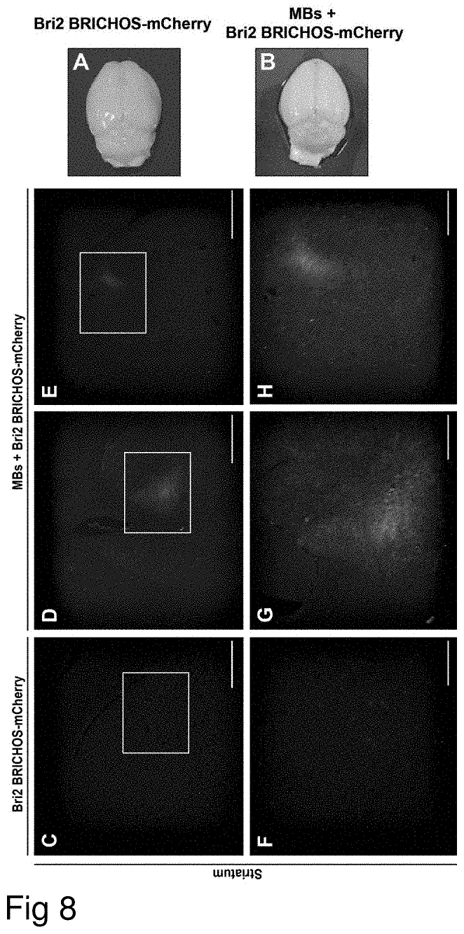

FIG. 8 shows that Rh Bri2 BRICHOS-mCherry (SEQ ID NO: 12) is found in the striatum after systemic injection in combination with lipid microbubbles. (A-B) Photographs of mouse brains after injection of rh Bri2 BRICHOS-mCherry only (A) or in combination with microbubbles (B). (C-H) Fluorescence of striatum regions collected 2 hours after injection of (C,F) 20 mg/kg rh Bri2 BRICHOS-mCherry only, and (D-E, G-H) microbubbles and 20 mg/kg rh Bri2 BRICHOS-mCherry. Sizes of scale bars are 200 μm for panels C-E, and 100 μm for panels F-H. Areas marked with white rectangles in C,D,E panels are enlarged in the F,G,H panels, respectively.

The brains of thoroughly perfused mice injected with either rh Bri2 BRICHOS-mCherry alone (one mouse) or microbubbles and rh Bri2 BRICHOS-mCherry (two mice) collected 2 hours after injections revealed a clear colour (white colour in FIG. 8) in the outer layer of the cortex and in the cerebellum only in the mice with rh Bri2 BRICHOS-mCherry plus microbubbles (FIG. 8A, B). Moreover, fluorescence (white colour in FIG. 8) was detected in the striatum region in paraffin embedded sections of the two mice injected with microbubbles and rh Bri2 BRICHOS-mCherry (FIG. 8D, E, G, H) but was not seen the mouse injected with rh Bri2 BRICHOS-mCherry alone (FIG. 8C, F). The data strongly suggests that intravenously injected rh Bri2 BRICHOS-mCherry passes into the brain parenchyma when microbubbles are injected into the circulation.

Further Experiments with Administration of Bri2 BRICHOS

### Methods

### Cell Culture

hCMEC/D3 cells (MilliporeSigma, Burlington, Mass.) were cultivated in EndoGRO-MV Complete Media (MilliporeSigma, Burlington, Mass.), supplemented with 1 ng/ml recombinant human fibroblast growth factor basic (FGF-2, Gibco, Invitrogen, Waltham, Mass.) and penicillin-streptomycin (100 IU/ml, 100 μg/ml, Gibco, Thermo Fisher Scientific, Waltham, Mass.) in T75 tissue culture flasks (TC-coated, DeltaLab, Spain) coated with 5-10 μg/cm² rat tail collagen type I (MilliporeSigma, Burlington, Mass.). The human brain microvascular endothelial cell line hCMEC/D3 is used as a human blood-brain barrier (BBB) in vitro model which produces an accurate in vivo phenotype, is reproducible and simply maintained. The hCMEC/D3 cell line has preserved the expression of majority of the transporters and receptors inherent to the BBB and therefore is efficiently used for studying the transport kinetics and mechanisms over the BBB. The hCMEC/D3 cell line has been previously described for the transport across the BBB for Aβ40 peptide, transthyretin and for several drug candidates.

Human neuroblastoma (SH-SY5Y) cell line (ATCC, Manassas, Va.) was grown in Dulbecco's Modified Eagle's Medium (DMEM, PAN-Biotech, Germany) with 4.5 g/L glucose, glutamine (2 mM) and 3.7 g/L NaHCO₃, supplemented with 10% Fetal Bovine Serum (FBS, Sera Plus, PAN-Biotech, Germany) and penicillin-streptomycin (100 IU/ml, 100 μg/ml) in TC-coated T75 tissue culture flasks. The human neuroblastoma cell line SH-SY5Y is a thrice-subcloned cell line derived from the SK-N-SH neuroblastoma cell line. It is widely used an in vitro neuronal model for studies of neurodegenerative diseases.

Human embryonic kidney 293 (HEK-293) cell line (ATCC, Manassas, Va.) was grown in Eagle's Minimum Essential Medium (EMEM, ATCC, Manassas, Va.), containing 2 mM L-glutamine, 1 mM sodium pyruvate and 1.5 g/L NaHCO₃, supplemented with 10% FBS and penicillin-streptomycin (100 IU/ml, 100 μg/ml) in TC-coated T75 tissue culture flasks.

Cultures were maintained in a humidified atmosphere (5% CO₂/95% air) at 37° C. and media was changed every other day. At approximately 80% confluency cells were trypsinated (0.025% trypsin, 0.01% EDTA, Gibco, Thermo Fisher Scientific, Waltham, Mass.) and passaged (until passage 35 for the hCMEC/D3 and SH-SY5Y cell lines).

hCMEC/D3 Monolayer Preparation

For the preparation of the hCMEC/D3 cell monolayers, transwell ethylene terephthalate (PET) membrane inserts (24-well, clear, pore size 0.4 μm, CellQART, SABEU GmbH & Co. KG, Germany) were coated with rat tail type I collagen (150 μg/ml in 1× Dulbecco's Phosphate-buffered saline pH 7.4, 1×DPBS, Gibco, Thermo Fisher Scientific, Waltham, Mass.) for 2 h at 37° C. Excess collagen was removed and the inserts were washed twice with 1×DPBS. hCMEC/D3 cells were seeded onto the freshly prepared collagen-coated inserts at a density of 50,000 cells/cm² and maintained in a humidified atmosphere (5% CO₂/95% air) at 37° C. Media was changed every second day and the cell monolayer was established by day 6.

Analysis of hCMEC/D3 Monolayer Integrity

The integrity of the monolayer was analyzed by crystal violet staining. The expression of the tight junction proteins zonula occludens-1 (ZO-1), junctional adhesion molecule A (JAM-A) and claudin-5 was confirmed by western blot and immunostaining. For crystal violet staining the cells were fixed with 3.7% formaldehyde (Sigma-Aldrich, St. Louis, Mo.) at room temperature for 2 min and permeabilized with 100% methanol (Sigma-Aldrich, St. Louis, Mo.) at room temperature for 20 min, after which the cells were stained with 0.01% crystal violet (Sigma-Adrich, St. Louis, Mo.) in 2% ethanol and analyzed using a fluorescence microscope (Axioskop 40, Zeiss, Germany). Images were captured by fluorescence microscopy using green (FITC/488) and red (TexasRed/570) channels.

For determination of the tight junction proteins, hCMEC/D3 cells were seeded onto collagen-coated 12-well plates (TC-coated, Cellstar, Greiner Bio-One, Austria) with at a seeding density of 50,000 cells/cm² and maintained in a humidified atmosphere (5% CO₂/95% air) at 37° C., changing the media every second day. After 6 days the cells were removed from the plate by scraping in ice cold 1×PBS, centrifuged at 14,000×g, 4° C. for 10 min, lysed in radioimmunoprecipitation assay (RIPA) buffer (Sigma, St. Louis, Mo.) containing 1 mM phenylmethylsulfonyl fluoride (PMSF, Thermo Scientific, Waltham, Mass.) and 1× Complete Protease Inhibitor (Roche, Switzerland), and then sonicated at 50% amplitude for 10 sec and incubated on ice for 20 min. Cell lysates were cleared by centrifugation at 14,000×g, 4° C. for 15 min and the total amount of protein in the cell lysates was determined by Bicinchoninic acid (BCA) assay (Pierce, Thermo Fisher Scientific, Waltham, Mass.), following manufacturers protocol. For western blots (see below) anti-Claudin-5, anti-JAM-A and anti-ZO-1 polyclonal rabbit primary antibodies (Invitrogen, Thermo Fisher Scientific, Waltham, Mass.), were used at 1:1000 dilution.

Analysis of hCMEC/D3 Monolayer Permeability

Experiments were conducted on culture day 6 by adding different BRICHOS protein constructs, or relevant controls to the apical side of the hCMEC/D3 monolayer. BRICHOS constructs used were rh proSP-C BRICHOS wt, rh proSP-C BRICHOS T187R, rh Bri2 BRICHOS wt monomers and oligomers, rh Bri2 BRICHOS R221E monomers, S-tag-Bri2 BRICHOS monomers and oligomers, mCherry-Bri2 BRICHOS monomers, NT-Bri2 BRICHOS monomers and oligomers and an unresolved mixture of different S-tag-Bri3 BRICHOS assembly states. Controls used were mCherry, NT*-tag, rh Aβ42 and apolipoprotein A-I (purified from human plasma with >98% purity; Chemicon, Sigma-Aldrich, St. Louis, Mo.). Concentrations used were 1 μM, 0.5 μM, 0.25 μM, 0.1 μM or 0.05 μM; and incubation times were either 2 h or 24 h. Monolayers were incubated in a humidified atmosphere (5% CO₂/95% air) at 37° C. After the incubation, medium on the apical and basolateral sides of the monolayer was removed and analyzed by Western blotting and the band intensities measured by ImageJ. For the western blot (see below) anti-Bri2 BRICHOS primary antibody (produced in goat and purified in house) in 1:1000 dilution, anti-proSP-C BRICHOS primary antibody (produced in goat and purified in house) at 1:2000 dilution, anti-S-tag primary antibody (S-tag protein HRP conjuc. Novagen, Merck, Germany) at 1:5000 dilution, anti-NT primary antibody (produced in rabbit and purified in house at 1:5000 dilution, anti-RFP primary antibody (produced in mouse, Thermo Fisher Scientific, Waltham, USA) at 1:1000 dilution and anti-human Apolipoprotein A-I primary antibody (produced in goat, Calbiochem, San Diego, Calif.) were used. For mCherry and mCherry-Bri2 BRICHOS monomers, fluorescence intensity of the apical and basolateral medium was recorded at λₑₓ 570 nm and λₑₘ 610 nm, after which the cells were fixed and analyzed by fluorescence microscopy to analyse the uptake of mCherry or mCherry-Bri2 BRICHOS into cells.

The permeability of the monolayer was also analyzed for fluorescein isothiocyanate dextran (FITC-dextran, Thermo Fisher Scientific, Waltham, Mass.) as negative control and human recombinant insulin (Sigma-Aldrich, St. Louis, Mo.) as positive control. For determining the FITC-dextran, insulin and propidium iodide permeability, 1 μM solutions in cell culture media were applied to the apical side of the monolayer and incubated in a humidified atmosphere (5% CO₂/95% air) at 37° C. for 24 h, after which the media from the apical and basolateral sides was removed. FITC fluorescence was measured at λₑₓ 492 nm and λₑₘ 518 nm. Insulin permeability was determined by western blot and an anti-Insulin monoclonal primary antibody (Sigma, St. Louis, Mo.) at 1:1000 dilution.

### Cell Viability

Cell viability was assessed by tetrazolium-based Thiazolyl Blue Tetrazolium Bromide (MTT) assay. For the MTT assay, 50 000 cells/cm² were seeded onto clear 96-well plates (TC, Cellstar, Greiner Bio-One, Austria) and grown until confluent, after which the cells were treated with the rh BRICHOS domains in 8 replicates for 24 h in a humidified atmosphere (5% CO₂/95% air) at 37° C. Treated cells were incubated with 0.5 mg/ml MTT (Sigma-Aldrich, St. Louis, Mo.) solution in FBS-free cell media for 4 h in a humidified atmosphere (5% CO²/95% air) at 37° C. The MTT solution was removed and the formazan crystals were dissolved in Dimethyl sulfoxide (DMSO, Sigma-Aldrich, St. Louis, Mo.) and incubated with shaking for 15 min. Absorbance was measured at 570 nm using a FLUOstar OPTIMA microplate reader (BMG Lab Tech, Germany). Untreated cells were used as positive control, and media without cells was used as a negative control. Also 2 μM Staurosporine from Streptomyces sp. (SP, Sigma-Aldrich, St. Louis, Mo.) was used as a negative control.

### Cell Uptake

For the cell uptake analysis, hCMEC/D3 cells were seeded onto collagen-coated 12-well plates and non-differentiated SH-SY5Y cells were seeded onto 12-well plates without collagen treatment, at a seeding density of 75,000 cells/cm² and maintained in a humidified atmosphere (5% CO₂/95% air) at 37° C., changing the media every second day. Once confluent, the hCMEC/D3 cells were treated with 1 μM rh Bri2 BRICHOS monomers, oligomers or proSP-C monomers for 2 h and 24 h. The SH-SY5Y cells were treated with either rh Bri2 BRICHOS monomers in concentrations 1 μM, 0.5 μM, 0.25 μM, 0.1 μM, 0.05 μM for 24 h; rh Bri2 BRICHOS monomers in concentrations 1 μM and 0.25 μM for 2 h and 24 h; rh Bri2 BRICHOS oligomers in 1 μM concentration for 24 h; or 0.25 μM and 1 μM rh proSP-C for 2 h and 24 h. Untreated cells were used as control. After the treatment, the cells were lysed as described for the HEK-293 cells. For the Western blot (see below), anti-Bri2 BRICHOS primary antibody (produced in goat and purified in house) was used at 1:1000 dilution, and anti-proSP-C BRICHOS primary antibody (produced in goat and purified in house) was used at 1:2000 dilution.

### Western Blot

Samples were separated by Tris-glycine SDS-PAGE, using 12% separating gel, under reducing conditions, including 5% β-Mercaptoethanol (Sigma, St. Louis, Mo.) and 5 mM DTT in the sample buffer and were heated at 95° C. for 5 min. Spectra Multicolor Broad Range protein ladder was used (Thermo Fisher Scientific, Waltham, USA). For the monolayer permeability assessment, 4 μl of protein sample in cell culture media was loaded in the wells. For the cell uptake assessment, 7 μg of cell lysate was loaded.

Electrophoresis was conducted at 120 V, and electrotransfer to a Polyvinylidene fluoride (PVDF) membrane (0.2 μm, Amersham Hybond, GE Healthcare, Chicago, Ill.) was achieved at 15 V for 30 min (Semi-dry transfer cell, Bio-Rad, Hercules, Calif.) with a transfer buffer formulation of 25 mM Tris-HCl pH 8.3, 192 mM glycine, 0.05% SDS, 20% methanol.

The membranes were soaked in blocking buffer [5% nonfat dry milk (AppliChem, Germany) diluted in 1×PBS containing 0.1% Tween-20 (Sigma, St. Louis, Mo.) (1×PBS-T)] for 30 min at room temperature and incubated with the primary antibody overnight at 4° C. Membranes were washed 3 times with 0.5% nonfat dry milk in 1×TPBS-T and incubated with horseradish peroxidase conjugated secondary antibody (Goat Anti-Rabbit IgG from abcam, UK in 1:3000 dilution; Rabbit Anti-Goat IgG from Invitrogen, Waltham, Mass. at 1:10000 dilution; Rabbit Anti-Mouse IgG from Invitrogen, Waltham, Ma at 1:10000 dilution; Goat Anti-Human IgG Fc from Invitrogen, Waltham, Ma in 1:1000 dilution) for 1 h at room temperature. Membranes were washed again as above, and also twice with 1×PBS. Blots were developed using chemiluminescent detection system (Pierce ECL plus, Thermo Fisher Scientific, Waltham, USA). The intensities were assessed using ImageJ software.

For insulin, extra experimental procedures were added. The blotted membrane was soaked with blocking buffer [1% nonfat dry milk and 0.1% BSA in TBS-T (TBS [50 mM Tris-HCl at pH 7.4, 150 mM NaCl] containing 0.1% Tween 20)] for 5 min and washed with PBS-T for 3 min. The membrane was incubated in 0.2% Glutaraldehyde in PBS-T for 15 min and washed 3 times with PBS-T, after which it was immersed in citrate retrieval buffer (10 mM citric acid pH 6.0, 1 mM EDTA, 0.05% Tween 20) and microwaved for 10 min at 600 W after boiling. After cooling to room temperature, the membrane was soaked with quenching buffer (200 mM glycine in PBS-T) for 10 min. After these extra steps, blotting was performed as described above.

### Analysis of NT-BRICHOS Fusion Protein Passage Over BBB in Wildtype Mice

A gene fragment encoding rh NT*-Bri2 BRICHOS fusion protein, where NT* is a solubility tag (as described in WO 2017/081239 A1) followed by human Bri2 residues 113-231, was cloned and expressed. The protein was expressed in Shuffle T7 competent E. coli cells that were grown in Lysogeny Broth (LB) medium supplemented with 15 μg/mL kanamycin at 30° C. When OD_{600 nm} reached ˜0.9, the temperature was lowered to 20° C. and over-night protein expression was induced by addition of 0.5 mM isopropyl b-D-1-thiogalactopyranoside (IPTG). Cells were harvested by centrifugation (3,000×g, 4° C.) and cell pellets were re-suspended in 20 mM Tris-HCl pH 8.0 followed by 5 min sonication (2 s on 2 s off, 65% power, on ice). The lysate was centrifuged (24,000×g, 4° C.) for 30 min, and the supernatant containing the target protein was then purified with an immobilized metal affinity chromatography (IMAC) column (Ni Sepharose™ 6 Fast Flow; GE Healthcare, UK) equilibrated with 20 mM Tris-HCl pH 8.0. The fusion protein was eluted with 300 mM imidazole in 20 mM Tris-HC pH 8.0, and dialyzed (regenerated cellulose RC, 6-8 kDa membrane; Spectrum Lab) against 20 mM Tris-HCl pH 8.0 overnight in cold room.

All the animal handling and experimental procedures were carried out in the animal facility, Huddinge campus, Karolinska Institutet according to local ethical guidelines and approved by Sodra Stockholm's Djurförsöksetiska Nämnd (dnr S 6-15) and Linköping's animal ethical board (ID 855). Three months old C57BL/6NTac (Taconic, Denmark) mice and 11 months old C57BL/6J mice (Janvier labs, France) were kept under controlled humidity and temperature on a 12-hour light-dark cycle, group-housed (seven per cage) with food and water available ad libitum. 3 Mice received a single i.v. injection of NT*-Bri2 BRICHOS fusion protein, 10 mg/kg, or equal volume of PBS, into the lateral tail vein by using a 0.3 mL syringe with a 30-gauge needle. Before the injections the mice were placed in a single cage under a heat lamp for 5 min, to dilate the tail veins. The 3 months old mice were anesthetized with isoflurane and intracardially perfused with 40 mL of saline (0.9% NaCl) 2 h after the injections. 11 months old mice received single i.v. injections of NT*-Bri2 BRICHOS fusion protein 10 mg/kg, or an equal volume of PBS, anesthetized and perfused 2 h after the injections. Brains were quickly removed and snap-frozen in dry ice and stored at −80° C. until analysis.

Immunohistochemical staining for NT*-Bri2 BRICHOS fusion protein was performed in 5 μm thick coronal sections of paraffin-embedded mouse brain tissue. The sections were de-paraffinized in xylene and re-hydrated in graded alcohol series from 99% to 70%. Brain sections were pre-treated for antigen retrieval in DIVA Decloaker 1× solution (Biocare Medical) inside a pressure cooker (Biocare Medical) at 110° C. for 30 min. The slides were cooled down at room temperature (RT) for 30 min, then washed with Tris-buffered saline containing 0.05% Tween 20 (TBS-T) and incubated first with peroxidase blocking solution (Dako) for 5 min. After washing in TBS-T, brain sections were additionally blocked with Background punisher (Dako) for 10 min. Subsequently, the slides were incubated with a solution containing the primary anti-NT antiserum from rabbit diluted in DAKO (Agilent) antibody diluent, overnight at 4° C. After washing with TBS-T, the sections were incubated for 30 min at RT with secondary cocktail containing horseradish peroxidase (HRP)-conjugated goat anti-rabbit antibody. HRP immunoreactivity was detected by permanent green (Biosite) solution. The sections were counterstained in hematoxylin Mayer, de-hydrated, cleared in xylene and mounted with DEPEX mounting media (Merck). Images were acquired using a Nikon Eclipse E800 light microscope linked to a high-resolution camera and using a 20× objective and 40× objective.

### Results

Bri2 BRICHOS Accumulates in CNS after Intravenous Administration

Aβ precursor protein (App) knock-in mouse models (Saito T. et al. (2014) Nat Neurosci. 17, 661-663), both App^{NL-G-F} mice (that express human Aβ with the Arctic mutation) and App^{NL-F} mice (that express wildtype human Aβ) were treated with repeated intravenous injections of rh Bri2 BRICHOS R221E (SEQ ID NO: 13). After the end of 10-12 weeks of treatment, Bri2 BRICHOS amounts in the CNS were analysed.

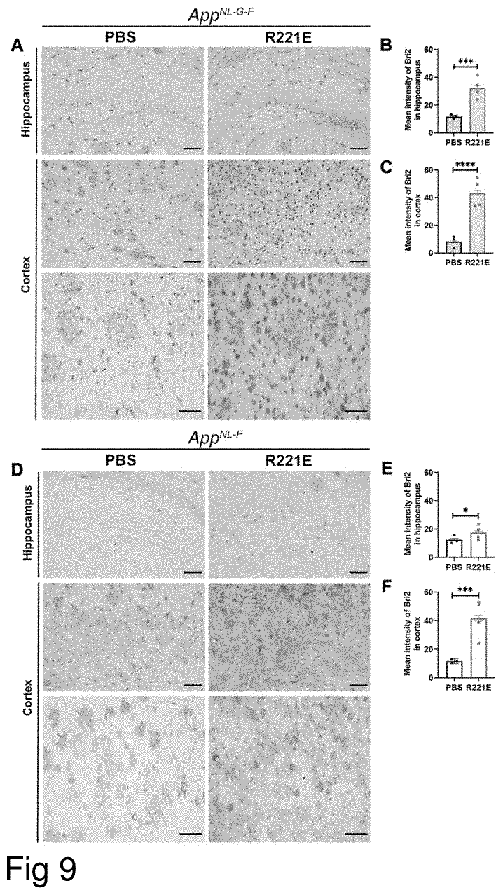

FIG. 9 shows Bri2 BRICHOS in App^{NL-G-F} and App^{NL-F} mouse brains after repeated injections of rh Bri2 BRICHOS R221E. Representative sections (A,D) were double stained with anti-Bri2 BRICHOS antibody (red/pink color) and 82E1 anti-Aβ antibody (green color) counterstained with hematoxylin Mayer (blue color) in rh Bri2 BRICHOS R221E or PBS treated App^{NL-G-F} and App^{NL-F} mice. Histograms represents mean intensity of Bri2 BRICHOS in hippocampus (B,E) and cortex (C,F) of App^{NL-G-F} (B,C) and App^{NL-F} (E,F) mice. Data are shown as mean±SEM (n=3-4 mice/group, 4 histological sections from each mouse analyzed). Unpaired parametric two-tailed t-test was used to calculate p-values. Scale bars represents 400 μm (upper four panels in A and D) and 100 μm (lower two panels in A and D). * p<0.05, ** p<0.01, *** p<0.001, **** p<0.0001.

The rh Bri2 BRICHOS R221E treated App knock-in mice showed more abundant overall Bri2 BRICHOS staining in the brain tissue including in neurons and around the Aβ plaques (FIG. 9, panels A and D) compared to the PBS treated control mice. There is a significant increase in overall Bri2 BRICHOS staining, analyzed from mean intensity of Bri2 BRICHOS between PBS and rh Bri2 BRICHOS R221E treated samples in both App^{NL-G-F} and App^{NL-F} mice (FIG. 9, panels B, C, E, F). Notably, the mice were sacrificed 2-4 weeks after the last dose of rh Bri2 BRICHOS R221E. These results strongly support that intravenously injected rh Bri2 BRICHOS R221E crosses the BBB in App knock-in AD mouse models and stays in the CNS parenchyma and cells for substantial time.

### Cell Viability

The effect of the rh BRICHOS domain on hCMEC/D3 cells and undifferentiated SH-SY5Y cells was assessed after 24 h incubation by tetrazolium-MTT cell viability assay. Rh Bri2 BRICHOS monomers show no toxicity to human brain microvascular endothelial cell line hCMEC/D3 and human neuroblastoma cell line SH-SY5Y, but a slight decrease in the cell mitochondrial activity in the presence of high concentration rh proSP-C BRICHOS and rh Bri2 BRICHOS oligomers or crude solution.

The effect of the rh Bri2 BRICHOS (113-231) wt monomers and oligomers on human cell lines hCMEC/D3 and SH-SY5Y is concentration dependent, with the concentration increase lowering the cell viability. For the rh Bri2 BRICHOS monomers in concentration range 1.0-0.1 μM, the SH-SY5Y cell viability increases from 94.0% to 99% and the hCMEC/D3 viability increases from 100.4% to 106.6%, with the rh Bri2 BRICHOS monomer lower concentrations slightly increasing hCMEC/D3 cell metabolic activity. Rh Bri2 BRICHOS oligomers at 1 μM concentration show 83.5% viability on SH-SY5Y cells and 86.7% viability on hCMEC/D3 cells. Rh proSP-C BRICHOS at 0.5 μM concentration show 88.9% viability on SH-SY5Y cells and at 1 μM concentration 90.3% viability on hCMEC/D3 cells. It is possible for the rh Bri2 BRICHOS oligomers at high concentration, like Aβ peptide, to commence a mild stress in the endoplasmic reticulum (ER) and Ca²⁺ release. In case of rh Bri2 BRICHOS monomers, the integrity of the cell membrane remains similar to the positive control. With the physiological concentration for the BRICHOS domain in human plasma being lower, it can be assumed that the BRICHOS domain is not harmful to the integrity of the BBB in normal conditions.

### Cell Uptake

Cell uptake for the rh BRICHOS domain was assessed for human cell lines SH-SY5Y and hCMEC/D3 after 2 h and 24 h incubation by western blotting and determined by band intensities.

24 h incubation of human neuroblastoma cell line SH-SY5Y with the rh Bri2 BRICHOS monomers in concentration ranging from 0.05 μM to 1 μM show a linear uptake. Bri2 BRICHOS oligomers are detected in the cell lysate to a lesser degree than the Bri2 BRICHOS, which may indicate the oligomers adhering to the cell surface. ProSP-C BRICHOS is similarly taken up by the SH-SY5Y cells, but to a lesser degree. No endogenous BRICHOS domain can be detected in the non-treated cells, showing the detectable BRICHOS domain is taken up from the cell media.

Cell uptake of different rh BRICHOS domain constructs and concentrations after 2 h and 24 h was also studied for the human brain microvascular endothelial cells hCMEC/D3. Similarly to the SH-SY5Y cells, Bri2 BRICHOS monomers and proSP-C BRICHOS are taken up by the hCMEC/D3 cells, but the Bri2 BRICHOS oligomers seem not to be taken up. No endogenous BRICHOS domain can be detected in the non-treated cells, showing the detectable BRICHOS domain is taken up from the cell media.

### Rh Bri2 BRICHOS Fused to Other Proteins is Efficiently Transcytosed Through a Human BBB Model In Vitro and Transported Over the BBB in Wildtype Mice

We used cultured human cerebral microvessel endothelial hCMEC/D3 cell monolayers as an in vitro model of the human BBB (Weksler, B. et al. (2013) Fluids Barriers CNS. 10, 16; and Markoutsa, E. et al. (2011) Eur J Pharm Biopharm. 77, 265-274) to investigate if rh Bri2 BRICHOS can be used as a transport vehicle to facilitate the CNS uptake of other proteins. We tested inter alia the passage of two unrelated proteins fused to rh Bri2 BRICHOS: mCherry, a ca 30 kDa fluorescent protein derived from sea anemones, and NT, a ca 15 kDa protein domain derived from spider silk.

The BRICHOS domain permeability was studied on the hCMEC/D3 cell line, after 6 days of seeding, when the tight junction monolayer had formed with high integrity. Rh Bri2 BRICHOS and proSP-C different constructs were assessed in different concentrations and incubation times and determined by band intensities.

Permeability studies of the recombinant human Bri2 BRICHOS (113-231) wild-type different quaternary structures show that monomers pass the hCMEC/D3 monolayer, but the oligomeric species do not pass. The permeability of the rh Bri2 BRICHOS monomers is time dependent and concentration dependant. Interestingly, Bri2 BRICHOS R221E monomer permeability is higher than that of Bri2 BRICHOS wt monomers. Rh proSP-C BRICHOS wt is determined to cross through the hCMEC/D3 monolayer less than the proSP-C BRICHOS T187R mutant.

TABLE 1
Permeability of different Bri2 and proSP-C BRICHOS domains after
2 h, 24 h or 48 h incubation. 1 μM BRICHOS solutions were
used. Results are represented as a mean of three repeats.

| | Time | | | |
|---|---|---|---|---|
| BRICHOS | point | BRICHOS concentration % (Mean ± SD) | | |
| construct | (h) | Apical | Basolateral | Monolayer |
| Bri2 BRICHOS | 2 | 92.1 ± 1.9 | 6.7 ± 1.4 | 1.2 ± 0.3 |
| monomers | 24 | 86.8 ± 1.8 | 10.4 ± 1.9 | 2.8 ± 0.1 |
| | 48 | 82.4 ± 2.1 | 14.5 ± 2.4 | 3.1 ± 0.3 |
| Bri2 BRICHOS | 24 | 100.0 ± 0.0 | 0.0 ± 0.0 | 0.0 ± 0.0 |
| crude | | | | |
| Bri2 BRICHOS | 24 | 100.0 ± 0.0 | 0.0 ± 0.0 | 0.0 ± 0.0 |
| oligomers | | | | |
| Bri2 BRICHOS | 24 | 80.6 ± 2.2 | 17.7 ± 1.8 | 1.7 ± 0.9 |
| R221E monomers | | | | |
| proSP-C | 24 | 93.7 ± 1.6 | 6 ± 1.8 | 0.3 ± 0.2 |
| BRICHOS wt | 48 | 92.3 ± 2.4 | 7.2 ± 2.5 | 0.5 ± 0.1 |
| proSP-C | 24 | 93.1 ± 3.6 | 6.3 ± 3.7 | 0.6 ± 0.1 |
| BRICHOS T187R | | | | |

TABLE 2
Rh Bri2 BRICHOS wild-type monomer hCMEC/D3 permeability
at different concentrationa and incubation times.
Results are represented as a mean of three repeats.

| Bri2 BRICHOS | | | | |
|---|---|---|---|---|
| monomer | | | | |
| concentration | Time point | BRICHOS concentration % (Mean ± SD) | | |
| (μM) | (h) | Apical | Basolateral | Monolayer |
| 1 | 3 | 90.9 ± 3.3 | 7.8 ± 1.8 | 1.3 ± 1.4 |
| | 24 | 88 ± 2.8 | 10.1 ± 2.3 | 1.9 ± 0.6 |
| 0.5 | 3 | 92.8 ± 3.0 | 5.9 ± 1.3 | 1.3 ± 0.2 |
| | 24 | 89.8 ± 2.0 | 8.5 ± 0.6 | 1.7 ± 1.4 |
| 0.25 | 3 | 97.6 ± 1.8 | 1.2 ± 0.7 | 1.2 ± 1.1 |
| | 24 | 91.8 ± 3.0 | 6.8 ± 3.6 | 1.4 ± 0.6 |
| 0.1 | 3 | 98.6 ± 2.0 | 0.3 ± 0.3 | 1.1 ± 1.1 |
| | 24 | 94.7 ± 3.8 | 4.13 ± 2.3 | 1.2 ± 1.2 |

Permeability of the Bri2 and Bri3 BRICHOS solubility tag conjugates show similarity to unconjugated Bri2 BRICHOS constructs, with the monomeric species crossing the hCMEC/D3 monolayer, but the oligomers or the crude solution not.

NT-Bri2 BRICHOS fusion protein and mCherry-Bri2 BRICHOS fusion protein show the same tendency as the unconjugated Bri2 BRICHOS (113-231) wt and the S-tag fusion protein, where only the monomers show permeability. On the other hand, the NT-tag separately or the mCherry separately does not show any permeability, demonstrating the Bri2 BRICHOS domain's capability of transporting conjugated molecules through the hCMEC/D3 monolayer.

From the data, it is clear that the endothelial cell uptake and permeability for the BRICHOS domain depends on the quaternary structure, where the monomeric species passes through the hCMEC/D3 monolayer, but the oligomeric does not. The hCMEC/D3 cell line has previously been shown to be size-selective and having restricted permeability in the apical to basolateral direction, with the barrier formation being more efficient towards large molecules. Bri2 BRICHOS R221E monomers and proSP-C BRICHOS T187R monomers show higher permeability due to the formation of stable monomers in comparison to their wild-type counterparts.

TABLE 3
Collective table for 1 μM solutions permeability after 24 h incubation.
Results are represented as a mean of three or four repeats (SD).

| | Concentration % (Mean ± SD) | | |
|---|---|---|---|
| Molecule of interest | Apical | Basolateral | Monolayer |
| proSP-C BRICHOS wt | 93.7 ± 1.6 | 6.0 ± 1.8 | 0.3 ± 0.2 |
| proSP-C BRICHOS | 93.1 ± 3.6 | 6.3 ± 3.7 | 0.6 ± 0.1 |
| T187R mutant | | | |
| S-tag-Bri3 BRCHOS | 100.0 ± 0.0 | 0.0 ± 0.0 | 0.0 ± 0.0 |
| (112-230) crude | | | |
| S-tag-Bri2 BRICHOS | 84.9 ± 3.0 | 13.3 ± 2.2 | 1.8 ± 0.5 |
| (90-236) monomers | | | |
| S-tag-Bri2 BRICHOS | 100.0 ± 0.0 | 0.0 ± 0.0 | 0.0 ± 0.0 |
| (90-236) oligomers | | | |
| Bri2 BRICHOS | 86.8 ± 1.8 | 10.4 ± 1.9 | 2.8 ± 0.1 |
| (113-231) wt monomers | | | |
| Bri2 BRICHOS | 100.0 ± 0.0 | 0.0 ± 0.0 | 0.0 ± 0.0 |
| (113-231) wt crude | | | |
| Bri2 BRICHOS | 100.0 ± 0.0 | 0.0 ± 0.0 | 0.0 ± 0.0 |
| (113-231) wt oligomers | | | |
| Bri2 BRICHOS R221E | 80.6 ± 2.2 | 17.7 ± 1.8 | 1.7 ± 0.9 |
| mutant monomers | | | |
| NT-Bri2 BRICHOS | 78.3 ± 1.7 | 20.0 ± 1.6 | 1.7 ± 0.3 |
| monomers | | | |
| NT-Bri2 BRICHOS | 99.3 ± 0.5 | 0.0 ± 0.0 | 0.7 ± 0.5 |
| oligomers | | | |
| mCherry-Bri2 BRICHOS | 82.6 ± 2.8 | 7.3 ± 1.2 | 10.1 ± 1.6 |
| monomers | | | |
| NT*-tag | 98.6 ± 2.0 | 0.0 ± 0.0 | 1.0 ± 1.0 |
| mCherry | 97.0 ± 2.2 | 1.3 ± 1.2 | 1.8 ± 1.7 |
| Human insulin | 85.1 | 13.4 | 1.6 |
| Human apolipoprotein | 94.8 ± 1.4 | 3.7 ± 2.3 | 1.5 ± 0.6 |
| A-I | | | |
| Aβ40 ** | 81.5 ± 4.0 | 0.0 ± 0.0 | 18.5 ± 4.0 *** |
| Aβ42 ** | 73.3 ± 6.1 | 0.0 ± 0.0 | 26.7 ± 6.1 *** |
| FITC-dextran | 98.8 ± 1.2 | 1.2 ± 1.2 | 0.0 ± 0.0 |

** represents a 0.5 μM solution.
*** represents aggregation in the solution.

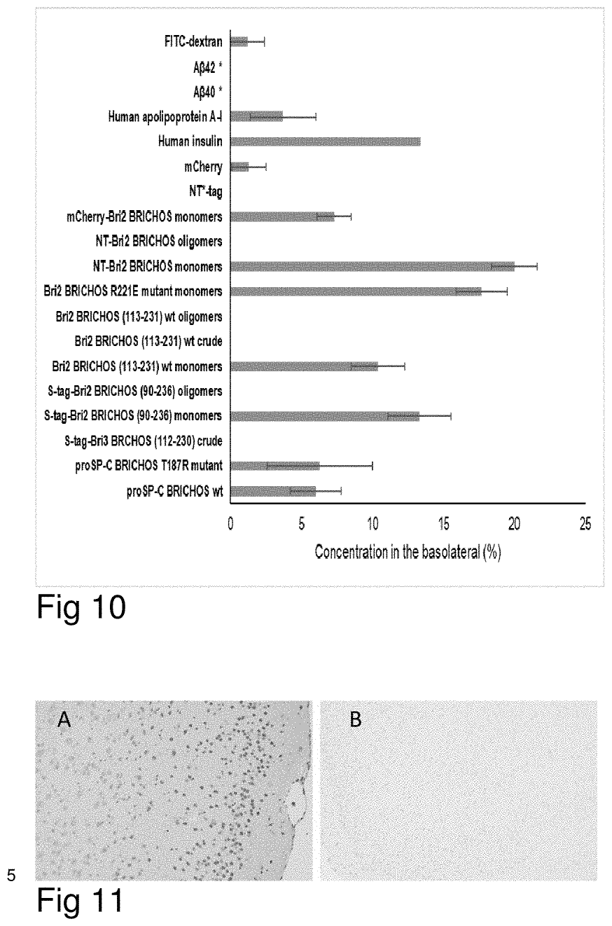

FIG. 10 and Table 3 show hCMEC/D3 monolayer permeability for 1 μM solutions of different proteins and macromolecules (in percent of total amount added to the apical side).

Both rh wildtype and R221E Bri2 BRICHOS monomers show significant passage from the apical side of the monolayers to the basolateral side, i.e. they are transcytosed, while rh wildtype Bri2 BRICHOS oligomers are not (FIG. 10). Neither mCherry nor NT alone passes through the monolayer to any substantial extent, but the fusion constructs with rh Bri2 BRICHOS domain were transcytosed in substantial quantities (FIG. 10).

Whether the substantial passage of Bri2 BRICHOS-mCherry over the human BBB model in vitro can be further increased by addition of microbubbles remains to be determined. And to what extent the human BBB model in vitro predicts all features of the mouse BBB in vivo remains to be determined.

To test whether rh Bri2 BRICHOS works as transport vehicle over the BBB in vivo, we repeated the same type of experiments as previously used to test the BBB passage of recombinant Bri2 and proSP-C BRICHOS in wildtype mice. FIG. 11 shows staining with anti-NT antibody (blue color) of brain cortex of mice injected with NT-Bri2 BRICHOS (A) or PBS (B). This showed that recombinant NT-Bri2 BRICHOS could be detected in brain parenchyma and in neuron cells two hours after intravenous injection, while PBS injected mice showed only background staining (FIG. 11).

These findings strongly support that the ability of rh Bri2 BRICHOS to pass the BBB can be harnessed to transport other proteins over the BBB, and that after passage into the CNS the fusion proteins spread in the parenchyma and are taken up into cells. Proteins generally do not pass the BBB passively, e.g. maximally 0.1% of peripherally administered antibodies pass the BBB (18), which indicates that an active mechanism mediates BBB transfer of rh Bri2 BRICHOS.

### Rh Bri2 BRICHOS Fused to a Target Protein is Endocytosed and Transported to Lysosomes in Primary Neurons.

The data in FIG. 11A indicate that the fusion protein rh NT-Bri2 BRICHOS is internalized in CNS cells and appears in intracellular vesicles. We studied the intracellular localization in more detail, using primary mouse neurons in culture and the recombinant fusion protein mCherry-Bri2 BRICHOS. Recombinant mCherry-Bri2 BRICHOS was added to the culture medium at low nanomolar concentrations, and after 24-48 h the neurons were immobilized and visualized under a fluorescence microscope.

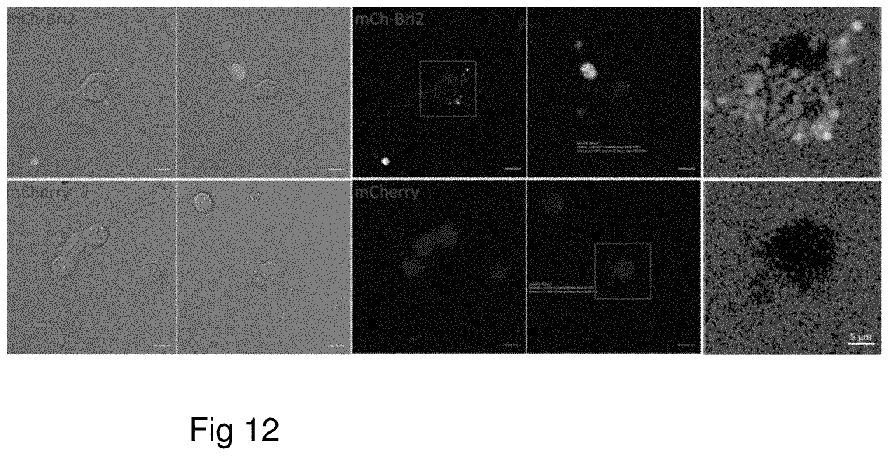

FIG. 12 shows that Rh Bri2 BRICHOS mediates uptake into mouse primary neurons. A fusion protein consisting of Bri2 BRICHOS linked to mCherry (denoted mCh-Bri2, results in upper row) is detected in abundant intracellular vesicles after addition to the culture medium. mCherry alone (results in lower row) is not taken up.

Thus, mCherry-Bri2 BRICHOS is endocytosed and can be detected in intracellular vesicles, while the control protein mCherry alone is not taken up to any detectable level. These data are in excellent agreement with the results from the transcytosis experiments in FIG. 10.

Finally, Rh Bri2 BRICHOS linked to a cargo protein is endocytosed and transported to lysosomes. Primary mouse neurons which were treated with the fusion protein mCherry-Bri2 BRICHOS show intracellular uptake and presence in lysosomes as shown by co-localization with the lysosome-specific dye SiR-Lyso (data not shown). Thus, labeling of the neurons that had been treated with mCherry-Bri2 BRICHOS with the lysosome-specific dye SiR-lysosome showed clear co-localization.

### Itemized List of Embodiments

1. An isolated recombinant protein selected from the group of proteins comprising an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); with the provisos that said protein is not comprising an amino acid sequence having at least 70% identity to residues 1-89 of Bri2 from human (SEQ ID NO: 3); and said protein is not comprising an amino acid sequence having at least 70% identity to human ABri23 (SEQ ID NO: 4); for use in a method of treatment of Alzheimer's disease in a mammal, including man, in need thereof comprising the steps of;
administrating to said mammal a plurality of lipid microbubbles and/or nanodroplets
administrating to said mammal said isolated recombinant protein;
   wherein said isolated recombinant protein is not comprised within said microbubbles and/or said nanodroplets; and
   wherein the method is not comprising any step of ultrasound treatment of any tissue of the mammal.
   2. An isolated recombinant protein selected from the group of proteins comprising an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); with the provisos that said protein is not comprising an amino acid sequence having at least 70% identity to residues 1-89 of Bri2 from human (SEQ ID NO: 3); and said protein is not comprising an amino acid sequence having at least 70% identity to human ABri23 (SEQ ID NO: 4); for use in a method of treatment of Alzheimer's disease in a mammal, including man, in need thereof consisting of the steps of;
administrating to said mammal a plurality of lipid microbubbles and/or nanodroplets
administrating to said mammal said isolated recombinant protein;
   wherein said isolated recombinant protein is not comprised within said microbubbles and/or said nanodroplets.
   3. A combination of an isolated recombinant protein and lipid microbubbles and/or nanodroplets according to any one of the preceding items for use in a method of treatment of Alzheimer's disease in a mammal, including man, in need thereof comprising the steps of;
administrating to said mammal a plurality of lipid microbubbles and/or nanodroplets
administrating to said mammal said isolated recombinant protein;
   wherein said isolated recombinant protein is not comprised within said microbubbles and/or said nanodroplets; and
   wherein the method is not comprising any step of ultrasound treatment of any tissue of the mammal.
   4. A combination of an isolated recombinant protein and lipid microbubbles and/or nanodroplets according to any one of the preceding items for use in a method of treatment of Alzheimer's disease in a mammal, including man, in need thereof consisting of the steps of;
administrating to said mammal a plurality of lipid microbubbles and/or nanodroplets
administrating to said mammal said isolated recombinant protein;
   wherein said isolated recombinant protein is not comprised within said microbubbles and/or said nanodroplets.
   5. An isolated recombinant protein or combination for use according to any one of the preceding items, wherein said isolated recombinant protein and said microbubbles and/or said nanodroplets are administered intravenously.
   6. An isolated recombinant protein or combination for use according to any one of items 1-5, wherein a therapeutically effective amount of at least 1 mg/kg, such as at least 5 mg/kg, more preferably such as at least 10 mg/kg an of said isolated recombinant protein is administered.
   7. An isolated recombinant protein or combination for use according to any one of items 1-5, wherein a therapeutically effective amount of less than 50 mg/kg, such as less than 30 mg/kg, more preferably less than 20 mg/kg of said isolated recombinant protein is administered.
   8. An isolated recombinant protein or combination for use according to any one of the items 1-7, wherein the isolated recombinant protein is selected from the group consisting of proteins comprising an amino acid sequence having at least 70%, preferably at least 75%, identity to residues 113-231 of Bri2 from human (SEQ ID NO: 2); and proteins comprising an amino acid sequence having at least 70%, preferably at least 75%, identity to the BRICHOS domain of Bri2 from human (SEQ ID NO: 5).
   9. An isolated recombinant protein or combination for use according to item 8, wherein the isolated recombinant protein is selected from the group consisting of proteins comprising an amino acid sequence having at least 80%, preferably at least 85%, identity to residues 113-231 of Bri2 from human (SEQ ID NO: 2); and proteins comprising an amino acid sequence having at least 80%, preferably at least 85%, identity to the BRICHOS domain of Bri2 from human (SEQ ID NO: 5).
   10. An isolated recombinant protein or combination for use according to item 9, wherein the isolated recombinant protein is selected from the group consisting of proteins comprising an amino acid sequence having at least 90%, preferably at least 95%, preferably at least 99%, identity to residues 113-231 of Bri2 from human (SEQ ID NO: 2); and proteins comprising an amino acid sequence having at least 90% identity to the BRICHOS domain of Bri2 from human (SEQ ID NO: 5).
   11. An isolated recombinant protein or combination for use according to item 10, wherein the isolated recombinant protein is selected from the group consisting of residues 113-231 of Bri2 from human (SEQ ID NO: 2); and the BRICHOS domain of Bri2 from human (SEQ ID NO: 5).
   12. An isolated recombinant protein or combination for use according to any one of items 1-7, wherein the isolated recombinant protein is selected from the group of proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
   13. An isolated recombinant protein or combination for use according to item 12, wherein the isolated recombinant protein is selected from the group of proteins comprising an amino acid sequence having at least 80%, preferably at least 85%, identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
   14. An isolated recombinant protein or combination for use according to item 13, wherein the isolated recombinant protein is selected from the group of proteins comprising an amino acid sequence having at least 90%, preferably at least 95%, identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
   15. An isolated recombinant protein or combination for use according to item 14, wherein the isolated recombinant protein is selected from the group consisting of any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
   16. An isolated recombinant protein or combination for use according to any one of the items 1-15, wherein, in the isolated recombinant protein, the amino acid residue corresponding to position 221 in SEQ ID NO: 1 is selected from the group consisting of Glu and Asp.
   17. An isolated recombinant protein or combination for use according to item 16, wherein, in the isolated recombinant protein, the amino acid residue corresponding to position 221 in SEQ ID NO: 1 is Glu.
   18. An isolated recombinant protein or combination for use according to any one of the items 1-17 wherein the isolated recombinant protein is consisting of less than or equal to 200 amino acid residues, such as less than or equal to 150 amino acid residues.
   19. An isolated recombinant protein or combination for use according to any one of items 1-18 wherein the isolated recombinant protein is consisting of more than or equal to 90 amino acid residues.
   20. An isolated recombinant protein or combination for use according to any one of the preceding items wherein said microbubbles and/or said nanodroplets are lipid coated.
   21. An isolated recombinant protein or combination for use according to any one of the preceding items, wherein said microbubbles and/or nanodroplets comprise 1,2-distearyol-sn-glycero-3-phosphocoline (DSPC), 1,2-distearyol-sn-glycero-3-phosphoethanolamine-N-(metoxy(polyethyleneglycol)2000) and a gas core of perfluorobutane.
   22. An isolated recombinant protein or combination for use according to any one of the items 1-21, wherein said microbubbles and/or nanodroplets comprise sulphur hexafluoride, polyethylene glycol (PEG, Macrogol), distearylphosphatidylcholine (DSPC), sodium 1,2-dipalmitoyl-sn-glycero phosphatidylglycerol and palmitic acid.
   23. An isolated recombinant protein or combination for use according to any one of the preceding items wherein said microbubbles and/or said nanodroplets have a diameter in the range of 1-8 μm, such as 2-6 μm, such as 4-5 μm.
   24. An isolated recombinant protein or combination for use according to any one of items 1-23, wherein said treatment is selected from the group consisting of preventive, palliative and curative treatment.
   25. A method of treating Alzheimer's disease in a mammal, including man, in need thereof comprising the steps of;
administrating to said mammal a plurality of lipid microbubbles and/or nanodroplets
administrating to said mammal an isolated recombinant protein selected from the group of proteins comprising an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); with the provisos that said protein is not comprising an amino acid sequence having at least 70% identity to residues 1-89 of Bri2 from human (SEQ ID NO: 3); and said protein is not comprising an amino acid sequence having at least 70% identity to human ABri23 (SEQ ID NO: 4);
   wherein said isolated recombinant protein is not comprised within said microbubbles and/or said nanodroplets; and
   wherein the method is not comprising any step of ultrasound treatment of any tissue of the mammal.
   26. A method of treating Alzheimer's disease in a mammal, including man, in need thereof consisting of the steps of;
administrating to said mammal a plurality of lipid microbubbles and/or nanodroplets
administrating to said mammal an isolated recombinant protein selected from the group of proteins comprising an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); with the provisos that said protein is not comprising an amino acid sequence having at least 70% identity to residues 1-89 of Bri2 from human (SEQ ID NO: 3); and said protein is not comprising an amino acid sequence having at least 70% identity to human ABri23 (SEQ ID NO: 4);
   wherein said isolated recombinant protein is not comprised within said microbubbles and/or said nanodroplets.
   27. A method according to any one items 25-26, wherein the isolated recombinant protein is selected from the group of proteins comprising an amino acid sequence having at least 70% identity to the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
   28. A method according to item 27, wherein the isolated recombinant protein is selected from the group of proteins comprising an amino acid sequence having at least 80%, preferably at least 85%, identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
   29. A method according to item 28, wherein the isolated recombinant protein is selected from the group of proteins comprising an amino acid sequence having at least 90%, preferably at least 95%, identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
   30. A method according to item 29, wherein the isolated recombinant protein is selected from the group of proteins comprising any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
   31. A method according to any one of the items 25-26, wherein the isolated recombinant protein is selected from the group of proteins comprising an amino acid sequence having at least 70% identity to any one of residues 113-231 of Bri2 from human (SEQ ID NO: 2) and the BRICHOS domain of Bri2 from human (SEQ ID NO: 5).
   32. A method according to item 31, wherein the isolated recombinant protein is selected from the group of proteins comprising an amino acid sequence having at least 80%, preferably at least 85%, identity to any one of residues 113-231 of Bri2 from human (SEQ ID NO: 2) and the BRICHOS domain of Bri2 from human (SEQ ID NO: 5).
   33. A method according to item 31, wherein the isolated recombinant protein is selected from the group of proteins comprising an amino acid sequence having at least 90%, preferably at least 95%, preferably at least 99% identity to any one of residues 113-231 of Bri2 from human (SEQ ID NO: 2) and the BRICHOS domain of Bri2 from human (SEQ ID NO: 5).
   34. A method according to item 33, wherein the isolated recombinant protein is selected from the group consisting of residues 113-231 of Bri2 from human (SEQ ID NO: 2); and the BRICHOS domain of Bri2 from human (SEQ ID NO: 5).
   35. A method according to any one of items 25-34, wherein the amino acid residue corresponding to position 221 in SEQ ID NO: 1 is selected from the group consisting of Glu and Asp.
   36. A method according to item 35, wherein the amino acid residue corresponding to position 221 in SEQ ID NO: 1 is Glu.
   37. A method according to any one of the items 25-34, wherein the lipid microbubbles and/or nanodroplets are as defined in any one of items 20-23.
   38. An isolated protein comprising
(i) a first protein moiety selected from the group of proteins comprising an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); and
(ii) a second protein or polypeptide moiety, preferably containing at least 50 amino acid residues;
   wherein said isolated protein is not comprising an amino acid sequence having at least 70% identity to residues 1-89 of Bri2 from human (SEQ ID NO: 3); and
   wherein said isolated protein is not comprising an amino acid sequence having at least 70% identity to human ABri23 (SEQ ID NO: 4).
   39. An isolated protein according to item 38, wherein the first protein moiety is selected from the group of proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
   40. An isolated protein according to item 39, wherein the first protein moiety is selected from the group of proteins comprising an amino acid sequence having at least 80%, preferably at least 85%, identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
   41. An isolated protein according to item 40, wherein the first protein moiety is selected from the group of proteins comprising an amino acid sequence having at least 90% identity, preferably at least 95%, to any one of the BRICHOS domains of Bri2 from human (SEQ ID NO: 5),chimpanzee (SEQ ID NO: 6), bovine (SEQ ID NO: 7), pig (SEQ ID NO: 8), mouse (SEQ ID NO: 9) and rat (SEQ ID NO: 10).
   42. An isolated protein according to item 41, wherein the first protein moiety is selected from the group of proteins comprising any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).
   43. An isolated protein according to item 38, wherein the first protein moiety is selected from the group of proteins comprising an amino acid sequence having at least 70% identity to any one of residues 113-231 of Bri2 from human (SEQ ID NO: 2) and the BRICHOS domain of Bri2 from human (SEQ ID NO: 5).
   44. An isolated protein according to item 43, wherein the first protein moiety is selected from the group of proteins comprising an amino acid sequence having at least 80%, preferably at least 85%, identity to any one of residues 113-231 of Bri2 from human (SEQ ID NO: 2) and the BRICHOS domains of Bri2 from human (SEQ ID NO: 5).
   45. An isolated protein according to item 44, wherein the first protein moiety is selected from the group of proteins comprising an amino acid sequence having at least 90%, preferably at least 95%, identity to any one of residues 113-231 of Bri2 from human (SEQ ID NO: 2) and the BRICHOS domain of Bri2 from human (SEQ ID NO: 5).
   46. An isolated protein according to item 45, wherein the first protein moiety is selected from the group consisting of residues 113-231 of Bri2 from human (SEQ ID NO: 2); and the BRICHOS domain of Bri2 from human (SEQ ID NO: 5).
   47. An isolated protein according to any one of items 38-46, wherein the amino acid residue in the first protein moiety corresponding to position 221 in SEQ ID NO: 1 is selected from the group consisting of Glu and Asp.
   48. An isolated protein according to item 47, wherein the amino acid residue in the first protein moiety corresponding to position 221 in SEQ ID NO: 1 is Glu.
   49. An isolated protein according to any one of items 38-48, wherein the first protein moiety is consisting of less than or equal to 200 amino acid residues, such as less than or equal to 150 amino acid residues.
   50. An isolated protein according to any one of items 38-48, wherein the first protein moiety is consisting of more than or equal to 90 amino acid residues.
   51. An isolated protein according to any one of items 38-50, wherein said second protein or polypeptide moiety contains from 50 to 2000 amino acid residues, such as from 50 to 1000 amino acid residues, such as from 50 to 500 amino acid residues, such as from 50 to 100 amino acid residues.
   52. An isolated protein according to any one of items 38-51, wherein the size of said second protein or polypeptide moiety is 5-200 kDa, such as 5-100 kDa, such as 5-50 kDa, such as 5-10 kDa.
   53. An isolated protein according to any one of items 38-52, wherein the first protein moiety is linked directly or indirectly to the amino-terminal or the carboxy-terminal end of the second protein or polypeptide moiety.
   54. An isolated protein according to any one of items 38-53, wherein the second protein or polypeptide moiety constitutes the amino-terminal and/or the carboxy-terminal end of the protein.
   55. An isolated protein according to any one of items 38-54, wherein the second protein or polypeptide moiety is selected from the group consisting of protein drugs, polypeptide drugs, protein tags, fluorescent proteins, antibodies, enzymes and/or neurotrophic factors.
   56. An isolated protein according to item 55, wherein the second protein or polypeptide moiety is an antibody.
   57. An isolated protein according to item 55, wherein the second protein or polypeptide moiety is a neurotrophin selected from the group consisting of brain-derived neurotrophic factor (BDNF), nerve growth factor (NGF), neurotrophin-3, neurotrophin-4, ciliary neurotrophic factor (CNTF), glial cell line-derived neurotrophic factor (GDNF), ephrins, epidermal growth factor (EGF), transforming growth factor (TGF), insulin-like growth factor (IGF), vascular endothelial growth factor (VEGF), fibroblast growth factor (FGF), platelet-derived growth factor (PDGF), and/or interleukins.
   58. An isolated protein according to item 55, wherein the second protein or polypeptide moiety is selected from the group consisting of α-L-iduronidase, Iduronate sulfatase, N-acetylgalactosamine 6-sulfatase, N-acetylgalactosamine 4-sulfatase, α-galactosidase, α-glucosidase, β-glucocerebrosidase and/or Lysosomal acid lipase.
   59. An isolated protein according to any one of the items 38-58, wherein said isolated protein is a recombinant fusion protein.
   60. An isolated protein according to any one of the items 38-58, wherein said first protein moiety is chemically linked to said second protein or polypeptide moiety.
   61. A combination of an isolated protein according to any one of the items 38-60 and a plurality of lipid microbubbles and/or nanodroplets, wherein said isolated protein is not comprised within said microbubbles and/or said nanodroplets.
   62. A kit comprising an isolated protein according to any one of the items 38-60 and a plurality of lipid microbubbles and/or nanodroplets, wherein said isolated protein is not comprised within said microbubbles and/or said nanodroplets.
   63. A combination or a kit according to any one of the items 61-62, wherein the lipid microbubbles and/or nanodroplets are as defined in any one of items 20-23.
   64. A method for transporting an isolated protein according to any one of items 38-60 across the blood-brain barrier in a mammal, including man, in need thereof consisting of the steps of;
administrating to said mammal a plurality of lipid microbubbles and/or nanodroplets;
administrating to said mammal said isolated protein.
   65. A method according to item 64, wherein the method is not comprising any step of ultrasound treatment of any tissue of the mammal.
   66. A method according to any one of the items 64-65, wherein the lipid microbubbles and/or nanodroplets are as defined in any one of items 20-23.
   67. An isolated protein according to any one of the items 38-60 for use in a method of treatment involving transporting said isolated protein across the blood-brain barrier in a mammal, including man, in need thereof comprising the steps of:
administrating to said mammal a plurality of lipid microbubbles and/or nanodroplets
administrating to said mammal said isolated protein;
   wherein said isolated protein is not comprised within said microbubbles and/or said nanodroplets.
   68. An isolated protein for use in the method according to item 67, wherein the method is not comprising any step of ultrasound treatment of any tissue of the mammal.
   69. An isolated protein according to any one of the items 38-60 for use in a method of treatment involving transporting said isolated protein across the blood-brain barrier in a mammal, including man, in need thereof consisting of the steps of:
administrating to said mammal a plurality of lipid microbubbles and/or nanodroplets
administrating to said mammal said isolated protein;
   wherein said isolated protein is not comprised within said microbubbles and/or said nanodroplets.
   70. An isolated protein for use according to any one of the items 67-69, wherein the lipid microbubbles and/or nanodroplets are as defined in any one of items 20-23.
   71. A method for transporting an isolated protein according to any one of items 38-60 across the blood-brain barrier in a mammal, including man, in need thereof comprising the step of:
administrating to said mammal said isolated protein.
   72. A method for transporting an isolated protein according to any one of items 38-60 across the blood-brain barrier in a mammal, including man, in need thereof consisting of the step of:
administrating to said mammal said isolated protein.
   73. A method of treating a medical condition in a mammal, including man, in need thereof comprising the step of:
administrating to said mammal an isolated protein according to any one of items 55-60.
   74. A method of treating a medical condition in a mammal, including man, in need thereof consisting of the step:
administrating to said mammal an isolated protein according to any one of items 55-60.
   75. An isolated protein according to any one of items 55-60 for use as a medicament.
   76. An isolated protein according to any one of items 55-60 for use in a method of treating a medical condition in a mammal, including man, in need thereof comprising the step of:
administrating to said mammal an isolated protein according to any one of items 55-60.
   77. An isolated protein according to any one of items 55-60 for use in a method of treating a medical condition in a mammal, including man, in need thereof consisting of the step of:
administrating to said mammal an isolated protein according to any one of items 55-60.

1. An isolated protein comprising
(i) a first protein moiety selected from the group of proteins comprising an amino acid sequence having at least 70% identity to residues 113-231 of Bri2 from human (SEQ ID NO: 2); and proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); and
(ii) a second protein or polypeptide moiety, preferably containing at least 50 amino acid residues;
wherein said isolated protein is not comprising an amino acid sequence having at least 70% identity to residues 1-89 of Bri2 from human (SEQ ID NO: 3); and
wherein said isolated protein is not comprising an amino acid sequence having at least 70% identity to human ABri23 (SEQ ID NO: 4).

2. The isolated protein according to claim 1, wherein the first protein moiety is selected from the group of proteins comprising an amino acid sequence having at least 70% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).

3. The isolated protein according to claim 2, wherein the first protein moiety is selected from the group of proteins comprising an amino acid sequence having at least 80% identity to any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10).

4. The isolated protein according to claim 3, wherein the first protein moiety is selected from the group of proteins comprising any one of the BRICHOS domains of Bri2 from human (SEQ ID NO: 5), chimpanzee (SEQ ID NO: 6), bovine (SEQ ID NO: 7), pig (SEQ ID NO: 8), mouse (SEQ ID NO: 9) and rat (SEQ ID NO: 10); or wherein the first protein moiety is selected from the group consisting of residues 113-231 of Bri2 from human (SEQ ID NO: 2); and the BRICHOS domain of Bri2 from human (SEQ ID NO: 5).

5. The isolated protein according to claim 1, wherein the amino acid residue in the first protein moiety corresponding to position 221 in SEQ ID NO: 1 is selected from the group consisting of Glu and Asp.

6. The isolated protein according to claim 5, wherein the amino acid residue in the first protein moiety corresponding to position 221 in SEQ ID NO: 1 is Glu.

7. The isolated protein according to claim 1, wherein the first protein moiety is consisting of less than or equal to 200 amino acid residues.

8. The isolated protein according to claim 1, wherein the first protein moiety is consisting of more than or equal to 90 amino acid residues.

9. The isolated protein according to claim 1, wherein said second protein or polypeptide moiety contains from 50 to 2000 amino acid residues.

10. The isolated protein according to claim 1, wherein the size of said second protein or polypeptide moiety is 5-200 kDa.

11. The isolated protein according to claim 1, wherein the second protein or polypeptide moiety is selected from the group consisting of protein drugs, polypeptide drugs, protein tags, fluorescent proteins, antibodies, enzymes and/or neurotrophic factors.

12. The isolated protein according to claim 11, wherein the second protein or polypeptide moiety is an antibody.

13. The isolated protein according to claim 11, wherein the second protein or polypeptide moiety is a neurotrophin selected from the group consisting of brain-derived neurotrophic factor (BDNF), nerve growth factor (NGF), neurotrophin-3, neurotrophin-4, ciliary neurotrophic factor (CNTF), glial cell line-derived neurotrophic factor (GDNF), ephrins, epidermal growth factor (EGF), transforming growth factor (TGF), insulin-like growth factor (IGF), vascular endothelial growth factor (VEGF), fibroblast growth factor (FGF), platelet-derived growth factor (PDGF), and/or interleukins.

14. The isolated protein according to claim 11, wherein the second protein or polypeptide moiety is selected from the group consisting of α-L-iduronidase, Iduronate sulfatase, N-acetylgalactosamine 6-sulfatase, N-acetylgalactosamine 4-sulfatase, α-galactosidase, α-glucosidase, β-glucocerebrosidase and/or Lysosomal acid lipase.

15. The isolated protein according to claim 11, wherein the second protein or polypeptide moiety is selected from the group consisting of enzymes known to be genetically mutated, and therefore functionally deficient in lysosomal storage diseases (LSDs).

16. The isolated protein according to claim 1, wherein said isolated protein is a recombinant fusion protein.

17. The isolated protein according to claim 1, wherein said first protein moiety is chemically linked to said second protein or polypeptide moiety.

18. A method for transporting the isolated protein according to claim 1 across the blood-brain barrier in a mammal, including man, in need thereof comprising the step of:
administrating to said mammal said isolated protein.

19. The method according to claim 18, further comprising the step of:
administrating to said mammal a plurality of lipid microbubbles and/or nanodroplets.

20. A method of treating a medical condition in a mammal, including man, in need thereof comprising the step of:
administrating to said mammal the isolated protein of claim 1.


SEQUENCE LISTING (14 sequences)

SEQ ID NO: 1 - protein, 266 aa, Homo sapiens
MVKVTFNSALAQKEAKKDEPKSGEEALIIPPDAVAVDCKDPDDVVPVGQRRAWCWCMCFGLAFMLAGVILGGAYLYKYFALQPDDVYYCGIKYIKDDVILNEPSADAPAALYQTIEENIKIFEEEEVEFISVPVPEFADSDPANIVHDFNKKLTAYLDLNLDKCYVIPLNTSIVMPPRNLLELLINIKAGTYLPQSYLIHEHMVITDRIENIDHLGFFIYRLCHDKETYKLQRRETIKGIQKREASNCFAIRHFENKFAVETLICS

SEQ ID NO: 2 - protein, 119 aa, Homo sapiens
QTIEENIKIFEEEEVEFISVPVPEFADSDPANIVHDFNKKLTAYLDLNLDKCYVIPLNTSIVMPPRNLLELLINIKAGTYLPQSYLIHEHMVITDRIENIDHLGFFIYRLCHDKETYKL

SEQ ID NO: 3 - protein, 89 aa, Homo sapiens
MVKVTFNSALAQKEAKKDEPKSGEEALIIPPDAVAVDCKDPDDVVPVGQRRAWCWCMCFGLAFMLAGVILGGAYLYKYFALQPDDVYYC

SEQ ID NO: 4 - protein, 23 aa, Homo sapiens
EASNCFAIRHFENKFAVETLICS

SEQ ID NO: 5 - protein, 95 aa, Homo sapiens
FADSDPANIVHDFNKKLTAYLDLNLDKCYVIPLNTSIVMPPRNLLELLINIKAGTYLPQSYLIHEHMVITDRIENIDHLGFFIYRLCHDKETYKL

SEQ ID NO: 6 - protein, 95 aa, Pan troglodytes; identical to SEQ ID NO: 5

SEQ ID NO: 7 - protein, 95 aa, Bos taurus
FADSDPANIVHDFNKKLTAYLDLNLDKCYVIPLNTSIVMPPKNLLELLINIKAGTYLPQSYLIHEHMVITDRIENIDHLGFYIYRLCHDKETYKL

SEQ ID NO: 8 - protein, 95 aa, Sus scrofa
FADSDPANIVHDFNKKLTAYLDLNLDKCYVIPLNTSIVMPPRNLLELLINIKAGTYLPQSYLIHEHMVITDRIENIDHLGFYIYRLCHDKETYKL

SEQ ID NO: 9 - protein, 95 aa, Mus musculus
FADSDPANIVHDFNKKLTAYLDLNLDKCYVIPLNTSIVMPPKNLLELLINIKAGTYLPQSYLIHEHMVITDRIENVDNLGFFIYRLCHDKETYKL

SEQ ID NO: 10 - protein, 95 aa, Rattus norvegicus
FADSDPANIVHDFNKKLTAYLDLNLDKCYVIPLNTSIVMPPRNLLELLINIKAGTYLPQSYLIHEHMVITDRIENVDHLGFFIYRLCHDKETYKL

SEQ ID NO: 11 - protein, 127 aa, Homo sapiens
  DOMAIN 3..121: note = Rh Bri2 BRICHOS domain
  DOMAIN 122..127: note = AU1 tag
GSQTIEENIKIFEEEEVEFISVPVPEFADSDPANIVHDFNKKLTAYLDLNLDKCYVIPLNTSIVMPPRNLLELLINIKAGTYLPQSYLIHEHMVITDRIENIDHLGFFIYRLCHDKETYKLDTYRYI

SEQ ID NO: 12 - protein, 363 aa, Homo sapiens
  DOMAIN 3..121: note = Rh Bri2 BRICHOS domain
  DOMAIN 129..363: note = mCherry
GSQTIEENIKIFEEEEVEFISVPVPEFADSDPANIVHDFNKKLTAYLDLNLDKCYVIPLNTSIVMPPRNLLELLINIKAGTYLPQSYLIHEHMVITDRIENIDHLGFFIYRLCHDKETYKLGGGGGGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 13 - protein, 119 aa, synthetic construct
QTIEENIKIFEEEEVEFISVPVPEFADSDPANIVHDFNKKLTAYLDLNLDKCYVIPLNTSIVMPPRNLLELLINIKAGTYLPQSYLIHEHMVITDRIENIDHLGFFIYELCHDKETYKL

SEQ ID NO: 14 - protein, 95 aa, synthetic construct
FADSDPANIVHDFNKKLTAYLDLNLDKCYVIPLNTSIVMPPRNLLELLINIKAGTYLPQSYLIHEHMVITDRIENIDHLGFFIYELCHDKETYKL